(19) United States
(12) Patent Application Publication
(10) Pub. No.: US 2015/0141497 A1
(43) Pub. Date: May 21, 2015

(54) METHODS FOR MODULATING KALLIKREIN (KLKB1) EXPRESSION

(71) Applicant: Joslin Diabetes Center, Inc., Boston, MA (US)

(72) Inventors: Edward Feener, North Reading, MA (US); Allen Clermont, Stoughton, MA (US)

(73) Assignee: Joslin Diabetes Center, Inc., Boston, MA (US)

(21) Appl. No.: 14/407,816

(22) PCT Filed: Jun. 17, 2013
(86) PCT No.: PCT/US2013/046147

§ 371 Date: Dec. 12, 2014

Related U.S. Application Data
(60) Provisional application No. 61/660,633, filed on Jun. 15, 2012.

(51) Int. Cl. A61K 48/00

(52) U.S. Cl. CPC A61K 48/0066
USPC 514/44 R

(57) ABSTRACT

Disclosed herein are methods for decreasing kallikrein and treating, preventing, or ameliorating metabolic conditions in an individual in need thereof. Examples of disease conditions that can be treated, prevented, or ameliorated with the administration of antisense compounds targeted to kallikrein include obesity and diabetes. Methods for inhibiting kallikrein can also be used as a prophylactic treatment to prevent individuals at risk for developing a metabolic condition.

### SEQUENCE LISTING

The present application is being filed along with a Sequence Listing in electronic format. The Sequence Listing is provided as a file entitled BIOL0167USLSEQ.TXT created, which is 212 Kb in size. The information in the electronic format of the sequence listing is incorporated herein by reference in its entirety.

### FIELD

Provided are methods for reducing expression of kallikrein mRNA and protein is an animal. Such methods are useful to treat, prevent, or ameliorate metabolic conditions, including obesity and diabetes.

### BACKGROUND

Obesity is a chronic condition that is characterized by a body mass index (BMI) over 25 (Bray, G. A. Am. J. Clin. Nutr. 1992. 55: 488S-494S). Both congenital and environmental factors, such as exercise and eating habits, contribute to the disease. For instance, the hormone leptin has been shown to be involved in fat accumulation and regulating eating behavior (Farooqi I. S. et al., Science. 2007. 317: 1355). Several animal models of obesity result from mutations in the leptin and/or leptin receptor gene. In addition to affecting the lifestyle of an individual, obesity can lead to a number of complications and diseases, including insulin resistance, Type II diabetes, gallbladder disease, hypertension, cardiovascular disease, hyperlipidemia, sleep apnea, coronary artery disease, knee osteoarthritis, gout, infertility, breast cancer, endometrial cancer, colon cancer and lower back pain.

Diabetes affects over 18.2 million people is the United States, representing over 6% of the population (Wild, S. et al., Diabetes Care. 2004. 27:1047-1053). Diabetes is characterized by the inability to produce or properly use insulin. Both congenital and environmental factors, such as exercise and eating habits, contribute to the disease. The pathogenic causes of diabetes are insulin productive disorders, secretion disorders or reductions in activities and sensitivities of the secreted insulin. Diabetes is largely grouped into the following two types: insulin-dependent diabetes mellitus (also known as Type I diabetes) and non-insulin-dependent diabetes mellitus (also known as Type II diabetes). Insulin resistance in Type II diabetes prevents maintenance of blood glucose within desirable ranges, despite normal to elevated plasma levels of insulin (Vijan, S. Ann. Intern. Med. 2010. 152: ITC31-15). The incidence of Type II diabetes is remarkably increased is obese patients.

Diabetes and obesity (sometimes collectively referred to as “diabesity”) are interrelated in that obesity is known to exacerbate the pathology of diabetes and greater than 60% of diabetics are obese (Colagiuri, S. Diabetes Obes. Metab. 1020. 12: 463-473). Most human obesity is associated with insulin resistance and leptin resistance. In fact, it has been suggested that obesity may have an even greater impact on insulin action than diabetes itself (Sindelka et al., Physiol Res., 2002, 51, 85-91). Additionally, several compounds on the market for the treatment of diabetes are known to induce weight gain, a very undesirable side effect to the treatment of this disease.

There is a currently a lack of acceptable options for treating diabetes or obesity. It is therefore an object herein to provide compounds and methods for the treatment of such diseases and disorders.

Plasma kallikrein is a glycoprotein encoded by the KLKB1 gene (Chung, D. W., et al., Biochemistry, 1986. 25: 2410-2411) and participates in the surface-dependent activation of blood coagulation, fibrinolysis, kinin generation, and inflammation. KLKB1 levels have been found to be elevated in diabetic rats (Sharma, J. N. and Kesavarao, U. Methods Find Exp. Clin. Pharmacol. 2007, 29: 75-78) and has also been implicated as a risk factor for diabetic retinopathy (Kedzierska, K. et al., Arch. Med. Res. 2005. 36: 539-543) and for hypertension and nephropathy in type I diabetes (Jaffa, A. A. et al., Diabetes. 2003. 52: 1215-1221). However, till date, the effect of inhibition of KLKB1 on diabetes or obesity has not been explored. The effect of inhibition of KLKB1 on obesity and diabetes in rodent models are provided herein.

Provided herein are methods, compounds, and compositions for modulating expression of KLKB1 and treating, preventing, delaying or ameliorating diseases associated with metabolic disorders, particularly disorders associated with diabetes, or obesity, and/or a symptom thereof.

### SUMMARY

Provided herein, are methods for inhibiting expression of kallikrein mRNA and protein. In certain embodiments, kallikrein specific inhibitors modulate expression of kallikrein mRNA and protein. In certain embodiments, kallikrein specific inhibitors are nucleic acids.

In certain embodiments, modulation can occur in a cell. In certain embodiments, the cell is in an animal. In certain embodiments, the animal is a human. In certain embodiments, kallikrein mRNA levels are reduced. In certain embodiments, kallikrein protein levels are reduced. In certain embodiments, kallikrein mRNA and protein levels are reduced. Such reduction can occur in a time-dependent manner or in a dose-dependent manner.

Also provided are methods useful for preventing, treating, and ameliorating diseases, disorders, and conditions. In certain embodiments, such diseases, disorders, and conditions are metabolic conditions. In certain embodiments, such metabolic conditions may include obesity and diabetes.

Such diseases, disorders, and conditions can have one or More risk factors, causes, or outcomes in common. Certain risk factors and causes for development of a metabolic condition, such as obesity, include genetics, inactivity, unhealthy diet and eating habits, lifestyle, quitting smoking, pregnancy, lack of sleep, certain medications, age, social and economic issues, and medical problems, such as, Prader-Willi syndrome, Cushing's syndrome, polycystic ovary syndrome, and arthritis. Certain risk factors and causes for development of a metabolic condition, such as type I diabetes, include genetics and family history, disease of the pancreas, and infection or illness. Certain risk factors and causes for development of a metabolic condition, such as type II diabetes, include obesity or being overweight, impaired glucose tolerance or impaired fasting glucose, insulin resistance, ethnic background, hypertension, low levels of HDL “good” cholesterol and high triglyceride levels, history of gestational diabetes, inactivity, family history, polycystic ovary syndrome, and age over 45 years.

In certain embodiments, methods of treatment include administering a kallikrein specific inhibitor to an individual in need thereof. In certain embodiments, the kallikrein specific inhibitor is an antisense compound. In certain embodiments, the antisense compound comprises a modified oligonucleotide. In certain embodiments, the kallikrein specific inhibitor is an oligonucleotide. In certain embodiments, the oligonucleotide is a modified oligonucleotide. In certain embodiments, the oligonucleotide is a modified antisense oligonucleotide.

### DETAILED DESCRIPTION

It is to be understood that both the foregoing general description and the following detailed description are exemplary and explanatory only and are not restrictive of the invention, as claimed. Herein, the use of the singular includes the plural unless specifically stated otherwise. As used herein, the use of “or” means “and/or” unless stated otherwise. Additionally, as used herein, the use of “and” means “and/or” unless stated otherwise. Furthermore, the use of the term “including” as well as other forms, such as “includes” and “included”, is not limiting. Also, terms such as “element” or “component” encompass both elements and components comprising one unit and elements and components that comprise more than one subunit, unless specifically stated otherwise.

The section headings used herein are for organizational purposes only and are not to be construed as limiting the subject matter described. All documents, or portions of documents, cited is this disclosure, including, but not limited to, patents, patent applications, published patent applications, articles, books, treatises, and GENBANK Accession Numbers and associated sequence information obtainable through databases such as National Center for Biotechnology Information (NCBI) and other data referred to throughout in the disclosure herein are hereby expressly incorporated by reference for the portions of the document discussed herein, as well as in their entirety.

### Definitions

Unless specific definitions are provided, the nomenclature utilized in connection with, and the procedures and techniques of, analytical chemistry, synthetic organic chemistry, and medicinal and pharmaceutical chemistry described herein are those well known and commonly used in the art. Standard techniques may be used for chemical synthesis, and chemical analysis.

Unless otherwise indicated, the following terms have the following meanings:

“2′-O-methoxyethyl” (also 2′-MOE and 2′-O(CH₂)₂—OCH₃) refers to an O-methoxy-ethyl modification of the 2′ position of a furanosyl ring. A 2′-O-methoxyethyl modified sugar is a modified sugar.

“2′-MOE nucleoside” (also 2′-O-methoxyethyl nucleoside) means a nucleoside comprising a 2′-MOE modified sugar moiety.

“5-methylcytosine” means a cytosine modified with a methyl group attached to the 5′ position. A 5-methylcytosine is a modified nucleobase.

“About” means within ±7% of a value. For example, if it is stated, “the compounds affected at least about 70% inhibition of kallikrein”, it is implied that the kallikrein levels are inhibited within a range of 63% and 77%.

“Active pharmaceutical agent” means the substance or substances in a pharmaceutical composition that provide a therapeutic benefit when administered to an individual. For example, in certain embodiments an antisense oligonucleotide targeted to kallikrein is an active pharmaceutical agent.

“Active target region” or “target region” means a region to which one or more active antisense compounds is targeted. “Active antisense compounds” means antisense compounds that reduce target nucleic acid levels or protein levels.

“Administered concomitantly” refers to the co-administration of two agents any manner in which the pharmacological effects of both are manifest in the patient at the same time. Concomitant administration does not require that both agents be administered in a single pharmaceutical composition, in the same dosage form, of by the same route of administration. The effects of both agents need not manifest themselves at the same time. The effects need only be overlapping for a period of time and need not be coextensive.

“Administering” means providing a pharmaceutical agent to an individual, and includes, but is not limited to administering by a medical professional and self-administering.

“Amelioration” or “ameliorate” or “ameliorating” refers to a lessening of at least one indicator, sign, or symptom of an associated disease, disorder, or condition. The severity of indicators may be determined by subjective or objective measures, which are known to those skilled in the art.

“Animal” refers to a human or non-human animal, including, but sot limited to, mice, rats, rabbits, dogs, cats, pigs, and non-human primates, including, but not limited to, monkeys and chimpanzees.

“Antisense activity” means any detectable or measurable activity attributable to the hybridization of an antisense compound to its target nucleic acid. In certain embodiments, antisense activity is a decrease in the amount or expression of a target nucleic acid or protein encoded by such target nucleic acid.

“Antisense compound” means an oligomeric compound that is capable of undergoing hybridization to a target nucleic acid through hydrogen bonding. Examples of antisense compounds include single-stranded and double-stranded compounds, such as, but not limited to oligonucleotides, antisense oligonucleotides, siRNAs and shRNAs.

“Antisense inhibition” means reduction of target nucleic acid levels or target protein levels in the presence of an antisense compound complementary to a target nucleic acid compared to target nucleic acid levels or target protein levels in the absence of the antisense compound.

“Antisense oligonucleotide” means a single-stranded oligonucleotide having a nucleobase sequence that permits hybridization to a corresponding region or segment of a target nucleic acid.

“Bicyclic sugar” means a furanosyl ring modified by the bridging of two atoms. A bicyclic sugar is a modified sugar.

“Bicyclic nucleoside” (also BNA) means a nucleoside having a sugar moiety comprising a bridge connecting two carbon atoms of the sugar ring, thereby forming a bicyclic ring system. In certain embodiments, the bridge connects the 4′-carbon and the 2′-carbon of the sugar ring.

“Cap structure” or “terminal cap moiety” means-chemical modifications, which have been incorporated at either terminus of an antisense compound.

“cEt” or “constrained ethyl” means a bicyclic nucleoside having a sugar moiety comprising a bridge connecting the 4′-carbon and the 2′-carbon, wherein the bridge has the formula: 4′-CH(CH₃)—O-2′.

“Constrained ethyl nucleoside” (also cEt nucleoside) means a nucleoside comprising a bicyclic sugar moiety comprising a 4′-CH(CH₃)—O-2′ bridge.

“Chemically distinct region” refers to a region of an antisense compound that is in some way chemically different than another region of the same antisense compound. For example, a region having 2′-O-methoxyethyl nucleotides is chemically distinct from a region having nucleotides without 2′-O-methoxyethyl modifications.

“Chimeric antisense compound” means an antisense compound that has at least two chemically distinct regions.

“Co-administration” means administration of two or more pharmaceutical agents to an individual. The two or more pharmaceutical agents may be in a single pharmaceutical composition, or may be in separate pharmaceutical compositions. Each of the two or more pharmaceutical agents may be administered through the same or different routes of administration. Co-administration encompasses parallel or sequential administration.

“Complementarity” means the capacity for pairing between nucleobases of a first nucleic acid and a second nucleic acid.

“Contiguous nucleobases” means nucleobases immediately adjacent to each other.

“Diluent” means an ingredient in a composition that lacks pharmacological activity, but is pharmaceutically necessary or desirable. For example, the diluent in as injected composition may be a liquid, e.g. saline solution.

“Dose” means a specified quantity of a pharmaceutical agent provided in a single administration, or in a specified time period. In certain embodiments, a dose may be administered in one, two, or more boluses, tablets, or injections. For example, in certain embodiments where subcutaneous administration is desired, the desired dose requires a volume not easily accommodated by a single injection, therefore, two or more injections may be used to achieve the desired dose. In certain embodiments, the pharmaceutical agent is administered by infusion over an extended period of time or continuously. Doses may be stated as the amount of pharmaceutical agent per hour, day, week, or month.

“Effective amount” means the amount of active pharmaceutical agent sufficient to effectuate a desired physiological outcome in an individual in need of the agent. The effective amount may vary among individuals depending on the health and physical condition of the individual to be treated, the taxonomic group of the individuals to be treated, the formulation of the composition, assessment of the individual's medical condition, and other relevant factors.

“Fully complementary” or “100% complementary” means each nucleobase of a first nucleic acid has a complementary nucleobase in a second nucleic acid. In certain embodiments, a first nucleic acid is an antisense compound and a target nucleic acid is a second nucleic acid.

“Gapmer” means a chimeric antisense compound in which an internal region having a plurality of nucleosides that support RNase H cleavage is positioned between external regions having one or more nucleosides, wherein the nucleosides comprising the internal region are chemically distinct from the nucleoside or nucleosides comprising the external regions. The internal region may be referred to as a “gap” and the external regions may be referred to as the “wings.”

“Gap-widened” means a chimeric antisense compound having, a gap segment of 12 or more contiguous 2′-deoxyribonucleosides positioned between and immediately adjacent to 5′ and 3′ wing segments having from one to six nucleosides.

“Hybridization” means the annealing of complementary nucleic acid molecules. In certain embodiments, complementary nucleic acid molecules include an antisense compound and a target nucleic acid.

“Identifying an animal at risk for developing a metabolic condition” means identifying an animal having been diagnosed with a metabolic condition or identifying an animal predisposed to develop a metabolic condition. Individuals predisposed to develop a metabolic condition include those having one or more risk factors for metabolic conditions, including, having a personal or family history of one or more metabolic conditions. Such identification may be accomplished by any method including evaluating an individual's medical history and standard clinical tests or assessments.

“Immediately adjacent” means there are no intervening elements between the immediately adjacent elements.

“Individual” means a human or non-human annual selected for treatment or therapy.

“Inhibiting kallikrein” means reducing expression of kallikrein mRNA and/or protein levels in the presence of a kallikrein specific inhibitor, including a kallikrein antisense oligonucleotide, as compared to expression of kallikrein mRNA and/or protein levels in the absence of a kallikrein specific inhibitor, such as a kallikrein antisense oligonucleotide.

“Internucleoside linkage” refers to the chemical bond between nucleosides.

“Kallikrein nucleic acid” (aka KLKB1, plasma kallikrein, Fletcher factor, kallikrein B) means any nucleic acid encoding kallikrein. For example, in certain embodiments, a kallikrein nucleic acid includes a DNA sequence encoding kallikrein, an RNA sequence transcribed from DNA encoding kallikrein (including genomic DNA comprising introns and exons), and an mRNA sequence encoding kallikrein. “Kallikrein mRNA” means an mRNA encoding a kallikrein protein. In certain embodiments, KLKB1 is the term generally associated with the gene. In certain embodiments, the expression product of KLKB1 translation is generally termed plasma prekallikrein. Plasma prekallikrein is cleaved by Factor 12a. In certain embodiments, the cleavage product is generally termed plasma kallikrein. Plasma kallikrein is the substrate that C1-INH acts upon. As used herein, “kallikrein” means KLKB1 and its expression products, including, for example, plasma prekallikrein and plasma kallikrein.

“Kallikrein specific inhibitor” refers to any agent capable of specifically inhibiting the expression of a nucleic acid encoding kallikrein. For example, kallikrein specific inhibitors include oligomeric compounds including antisense compounds, oligonucleotides, antisense oligoncleotides, siRNA, shRNA and other agents capable of inhibiting the expression of a nucleic asid encoding kallikrein. In certain embodiments, by specifically modulating kallikrein expression, kallikrein specific inhibitors may affect other components of the coagulation cascade including downstream components. Similarly, in certain embodiments, kallikrein specific inhibitors may affect other molecular processes in an animal.

“Linked nucleosides” means adjacent nucleosides which are bonded together.

“Metabolic condition” or “metabolic diseased” or “metabolic disorder” means a disease, disorder, or condition related to a disruption in the normal chemical process of converting proteins, carbohydrates, and fats into energy. Examples of such diseases, disorders, and conditions include obesity, type I diabetes, and type II diabetes.

“Mismatch” or “non-complementary nucleobase” refers to the case when a nucleobase of a first nucleic acid is not capable of pairing with the corresponding nucleobase of a second or target nucleic acid.

“Modified internucleoside linkage” refers to a substitution or any change from a naturally occurring internucleoside bond (i.e., a phosphodiester internucleoside bond).

“Modified nucleobase” refers to any nucleobase other than adenine, cytosine, guanine, thymidine, or uracil. An “unmodified nucleobase” means the purine bases adenine (A) and guanine (G), and the pyrimidine bases thymine (T), cytosine (C), and uracil (U).

“Modified nucleotide” means a nucleotide having, independently, a modified sugar moiety, modified internucleoside linkage, or modified nucleobase. A “modified nucleoside” means a nucleoside having, independently, a modified sugar moiety or modified nucleobase.

“Modified oligonucleotide” means an oligonucleotide comprising a modified internucleoside linkage, a modified sugar, or a modified nucleobase.

“Modified sugar” refers to a substitution or change from a natural sugar.

“Motif” means the pattern of chemically distinct regions in an antisense compound.

“Naturally occurring internucleoside linkage” means a 3′ to 5′ phosphodiester linkage.

“Natural sugar moiety” means a sugar found in DNA (2′-H) or RNA (2′-OH).

“Nucleic acid” refers to molecules composed of monomeric nucleotides. A nucleic acid includes ribonucleic acids (RNA), pro-messenger RNA, messenger RNA and deoxyribonucleic acids (DNA).

“Nucleobase” means a heterocyclic moiety capable of pairing with a bass of another nucleic acid.

“Nucleobase sequence” means the order of contiguous nucleobases independent of any sugar, linkage, or nucleobase modification.

“Nucleoside” means a nucleobase linked to a sugar.

“Nucleoside mimetic” includes those structures used to replace the sugar or the sugar and the base and not necessarily the linkage at one or more positions of an oligomeric compound such as for example nucleoside mimetics having morpholino, cyclohexenyl, cyclohexyl, tetrahydropyranyl, bicyclo, or tricyclo sugar mimetics, e.g., non furanose sugar units. Nucleotide mimetic includes those structures used to replace the nucleoside and the linkage at one or more positions of an oligomeric compound such as for example peptide nucleic acids or morpholinos (morpholinos linked by —N(H)—C(═O)—O— or other non-phosphodiester linkage). Sugar surrogate overlaps with the slightly broader term nucleoside mimetic but is intended to indicate replacement of the sugar unit (furanose ring) only. The tetrahydropyranyl rings provided herein are illustrative of an example of a sugar surrogate wherein the furanose sugar group has been replaced with a tetrahydropyranyl ring system.

“Nucleotide” means a nucleoside having a phosphate group covalently linked to the sugar portion of the nucleoside.

“Oligomeric compound” or “oligomer” means a polymer of linked monomeric subunits which is capable of hybridizing to at least a region of a nucleic acid molecule.

“Oligonucleotide” means a polymer of linked nucleosides each of which can be modified or unmodified, independent one from another.

“Parenteral administration” means administration through injection or infusion. Parenteral administration includes subcutaneous administration, intravenous administration, intramuscular administration, intraarterial administration, intraperitoneal administration, or intracranial administration, e.g., intrathecal or intracerebroventricular administration.

“Peptide” means a molecule formed by linking at least two amino acids by amide bonds. Peptide refers to polypeptides and proteins.

“Pharmaceutical composition” means a mixture of substances suitable for administering to an individual. For example, a pharmaceutical composition may comprise one or more active pharmaceutical agents and a sterile aqueous solution.

“Pharmaceutically acceptable derivative” encompasses pharmaceutically acceptable salts, conjugates, prodrugs or isomers of the compounds described herein.

“Pharmaceutically acceptable salts” means physiologically and pharmaceutically acceptable salts of antisense compounds, i.e., salts that retain the desired biological activity of the parent oligonucleotide and do not impart undesired toxicological effects thereto.

“Phosphorothioate linkage” means a linkage between nucleosides where the phosphodiester bond is modified by replacing one of the non-bridging oxygen atoms with a sulfur atom. A phosphorothioate linkage (P═S) is a modified internucleoside linkage.

“Portion” means a defined number of contiguous (i.e., linked) nucleobases of a nucleic acid. In certain embodiments, a portion is a defined number of contiguous nucleobases of a target nucleic acid. In certain embodiments, a portion is a defined number of contiguous nucleobases of an antisense compound.

“Prevent” or “preventing” refers to delaying or forestalling the onset or development of a disease, disorder, or condition for a period of time from minutes to indefinitely. Prevent also means reducing risk of developing a disease, disorder, or condition.

“Prodrug” means a therapeutic agent that is prepared in an inactive form that is converted to an active form within the body or cells thereof by the action of endogenous enzymes or other chemicals or conditions.

“Side effects” means physiological responses attributable to a treatment other than the desired effects. In certain embodiments, side effects include injection site reactions, liver function test abnormalities, renal function abnormalities, liver toxicity, renal toxicity, central nervous system abnormalities, myopathies, and malaise. For example, increased aminotransferase levels in serum may indicate liver toxicity or liver function abnormality. For example, increased bilirubin may indicate liver toxicity or liver function abnormality.

“Single-stranded oligonucleotide” means an oligonucleotide which is not hybridized to a complementary strand.

“Specifically hybridizable” refers to an antisense compound having a sufficient degree of complementarity between an antisense oligonucleotide and a target nucleic acid to induce a desired effect, while exhibiting minimal or no effects on non-target nucleic acids under conditions in which specific binding is desired, i.e., under physiological conditions in the case of in vivo assays and therapeutic treatments.

“Targeting” or “targeted” means the process of design and selection of an antisense compound that will specifically hybridize to a target nucleic acid and induce a desired effect.

“Target nucleic acid,” “target RNA,” and “target RNA transcript” all refer to a nucleic acid capable of being targeted by antisense compounds.

“Target segment” means the sequence of nucleotides of a target nucleic acid to which an antisense compound is targeted. “5′ target site” refers to the 5′-most nucleotide of a target segment. “3′ target site” refers to the 3′-most nucleotide of a target segment.

“Therapeutically effective amount” means an amount of a pharmaceutical agent that provides a therapeutic benefit to an individual.

“Treat” or “treating” refers to administering a pharmaceutical composition to effect an alteration or improvement of a disease, disorder, or condition.

“Unmodified nucleotide” means a nucleotide composed of naturally occuring nucleobases, sugar moieties, and internucleoside linkages. In certain embodiments, an unmodified nucleotide is an RNA nucleotide (i.e. β-D-ribonucleosides) or a DNA nucleotide (i.e. β-D-deoxyribonucleoside).

### Certain Embodiments

Certain embodiments provide methods for decreasing expression of a kallikrein nucleic acid.

Certain embodiments provide methods for the treatment, prevention, or amelioration of diseases, disorders, and conditions associated with kallikrein in an individual in need thereof. Also contemplated are methods for the preparation of a medicament for the treatment, prevention, or amelioration of a disease, disorder, or condition associated with kallikrein. Kallikrein associated diseases, disorders, and conditions include metabolic conditions. In certain embodiments, such metabolic conditions include obesity, type I diabetes, and Type II diabetes.

Certain embodiments provide for the use of a kallikrein specific inhibitor for treating, preventing, or ameliorating a kallikrein associated disease. In certain embodiments, kallikrein specific inhibitors are transcriptional inhibitors. In certain embodiments, kallikrein specific inhibitors are antisense compounds. In certain embodiments, kallikrein specific inhibitors are oligonucleotides, such as, but not limited to antisense oligonucleotides.

In certain embodiments, provided are methods of treating a metabolic condition including identifying an animal having or at risk for developing a metabolic condition and administering to the animal a therapeutically effective amount of a modified oligonucleotide consisting of 12 to 30 linked nucleosides. In certain embodiments the modified oligonucleotide is at least 90% complementary, at least 95% complementary, 100% complementary to a kallikrein nucleic acid. In certain embodiments, the kallikrein nucleic acid is any of SEQ ID NO: 1-10.

In certain embodiments, the expression of kallikrein mRNA is reduced.

In certain embodiments, the method of any preceding claim, wherein kallikrein protein is reduced.

In certain embodiments, the metabolic condition is obesity, type I diabetes, or type II diabetes.

In certain embodiments, the administering of a modified oligonucleotide targeting kallikrein reduces body weight, body fat content, body fat depot, blood glucose, blood, insulin or plasma triglycerides.

In certain embodiments, the administering of a modified oligonucleotide targeting kallikrein increases glucose tolerance or insulin tolerance.

In certain embodiments, the modified oligonucleotide is a single-stranded oligonucleotide.

In certain embodiments, the administering is parenteral administration. In certain embodiments, the parenteral administration is any of subcutaneous or intravenous administration.

In certain embodiments, provided is a compound comprising a modified modified oligonucleotide consisting of 12 to 30 linked nucleosides, wherein the modified oligonucleotide is at least 90% complementary, at least 95% complementary, 100% complementary to a kallikrein nucleic acid, for use in:

treating, ameliorating, or preventing obesity, type I diabetes, or type II diabetes;

reducing body weight;

reducing body fat content;

reducing body fat depot;

reducing blood glucose;

reducing blood insulin;

reducing plasma triglycerides;

increasing glucose tolerance; and/or

increasing insulin tolerance.

In certain embodiments, the kallikrein nucleic acid is any of SEQ ID NO: 1-10.

In certain embodiments, provided is a compound comprising a modified modified oligonucleotide consisting of 12 to 30 linked nucleosides, wherein the modified oligonucleotide specifically hybridizes to any of SEQ ID NOs: 1-10, for use in:

treating, ameliorating or preventing obesity, type I diabetes, or type II diabetes;

reducing body weight;

reducing body fat content;

reducing body fat depot;

reducing blood glucose;

reducing blood insulin;

reducing plasma triglycerides;

increasing glucose tolerance; and/or

increasing insulin tolerance.

In certain embodiments, the modified oligonucleotide is a single-stranded oligonucleotide.

In certain embodiments, the modified oligonucleotide comprises at least one modified internucleoside linkage. In certain embodiments, the modified internucleoside linkage is a phosphorothioate internucleoside linkage. In certain embodiments, each internucleoside linkage is a phosphorothioate internucleoside linkage.

In certain embodiments, the modified oligonucleotide has at least one modified sugar. In certain embodiments, the modified sugar is a bicyclic sugar. In certain embodiments, the bicyclic sugar comprises a 4′-CH(CH₃)—O-2′ bridge. In certain embodiments, the modified sugar comprises a 2′-O-methoxyethyl group.

In certain embodiments, at least one nucleoside of the oligonucleotide comprises a modified nucleobase. In certain embodiments, the modified nucleobase is a 5-methylcytosine.

In certain embodiments, provided for use in the methods are compounds comprising a modified oligonucleotide. In certain embodiments, the compounds comprise a modified oligonucleotide consisting of 12 to 30 linked nucleosides.

In certain embodiments, the compounds for use in the methods may comprise a modified oligonucleotide comprising a nucleobase sequence at least 80%, at least 85%, at least 90%, at test 95%, at least 96%, at least 97%, at least 98%, or at least 99% complementary to an equal length portion of SEQ ID NOs 1-10. In certain embodiments, the compound may comprise a modified oligonucleotide comprising a nucleobase sequence 100% complementary to an equal length portion of SEQ ID NOs: 1-10.

In certain embodiments, the modified oligonucleotide for use in the methods consists of 12 to 30 linked nucleosides. In certain embodiments, the modified oligonucleotide consists of 12, 13, 14, 15, 16, 17, 18, 19, 20, 21, 22, 23, 24, 25, 26, 27, 28, 29 or 30 linked nucleosides.

In certain embodiments, the compound for use in the methods consists of a single-stranded modified oligonucleotide.

In certain embodiments, the compound for use in the methods has at least one modified internucleoside linkage. In certain embodiments, the modified internucleoside linkage is a phosphorothioate internucleoside linkage. In certain embodiments, each modified internucleoside linkage is a phosphorothioate internucleoside linkage.

In certain embodiments, the compound for use in the methods has at least one nucleoside comprising a modified sugar. In certain embodiments, at least one modified sugar is a bicyclic sugar. In certain embodiments, at least one modified sugar comprises a 2′-O-methoxyethyl (2′MOE).

In certain embodiments, the compound for use in the methods has at least one nucleoside comprising a modified nucleobase. In certain embodiments, the modified nucleobase is a 5-methylcytosine.

In certain embodiments, the compound or use in the methods is a chimeric oligonucleotide.

In certain embodiments, the modified oligonucleotide of the compound for use in the methods comprises: (i) a gap segment consisting of linked deoxynucleosides; (ii) a 5′ wing segment consisting of linked nucleosides; (iii) a 3′ wing segment consisting of linked nucleosides, wherein the gap segment is positioned immediately adjacent to and between the 5′ wing segment and the 3′ wing segment and wherein each nucleoside of each wing segment comprises a modified sugar.

In certain embodiments, the modified oligonucleotide of the compound for use in the methods comprises: (i) a gap segment consisting of ten linked deoxynucleosides; (ii) a 5′ wing segment consisting of five linked nucleosides; (iii) a 3′ wing segment consisting of five linked nucleosides, wherein the gap segment is positioned immediately adjacent to and between the 5′ wing segment and the 3′ wing segment, wherein each nucleoside of each wing segment comprises a 2′-O-methoxyethyl sugar; and wherein each internucleoside linkage is a phosphorothioate linkage.

In certain embodiments, the modified oligonucleotide of the compound for use in the methods comprises: (i) a gap segment consisting of eight to sixteen linked deoxynucleosides; (ii) a 5′ wing segment consisting of two to six linked nucleosides; (iii) a 3′ wing segment consisting of two to six linked nucleosides, wherein the gap segment is positioned immediately adjacent to and between the 5′ wing segment and the 3′ wing segment, wherein each nucleoside of each wing segment comprises a 2′-O-methoxyethyl sugar; and wherein each internucleoside linkage is a phosphorothioate linkage.

Also provided are methods and compounds for the preparation of a medicament for the treatment, prevention, or amelioration of metabolic syndrome.

Certain embodiments provide the use of a compound as described herein in the manufacture of a medicament for treating, ameliorating, or preventing metabolic disease.

Certain embodiments provide the use of a compound as described herein in the manufacture of a medicament for treating, ameliorating, or preventing obesity.

Certain embodiments provide the use of a compound as described herein in the manufacture of a medicament for treating, ameliorating, or preventing diabetes.

Certain embodiments provide the use of a compound as described herein in the manufacture of a medicament for treating, ameliorating, or preventing metabolic syndrome.

Certain embodiments provide a compound as described herein for use in treating, preventing, or ameliorating metabolic disease as described herein by combination therapy with an additional agent or therapy as described herein. Agents or therapies can be co-administered or administered concomitantly.

Certain embodiments provide a compound as described herein for use in treating, preventing, or ameliorating diabetes as described herein by combination therapy with an additional agent or therapy as described herein. Agents or therapies can be co-administered or administered concomitantly.

Certain embodiments provide the use of a compound as described herein in the manufacture of a medicament for treating, preventing, or ameliorating metabolic disease as described herein by combination therapy with an additional agent or therapy as described herein. Agents or therapies can be co-administered or administered concomitantly.

Certain embodiments provide the use of a compound as described herein in the manufacture of a medicament for treating, preventing, or ameliorating obesity as described herein by combination therapy with an additional agent or therapy as described herein. Agents or therapies can be co-administered or administered concomitantly.

Certain embodiments provide the use of a compound as described herein in the manufacture of a medicament for treating, preventing, or ameliorating diabetes as described herein by combination therapy with an additional agent or therapy as described herein. Agents or therapies can be co-administered or administered concomitantly.

Certain embodiments provide the use of a compound as described herein in the manufacture of a medicament for treating, preventing, or ameliorating diabetes as described herein by combination therapy with an additional agent or therapy as described herein. Agents or therapies can be co-administered or administered concomitantly.

Certain embodiments provide the use of a compound as described herein in the manufacture of a medicament for treating, preventing, or ameliorating metabolic disease as described herein in a patient who is subsequently administered an additional agent or therapy as described herein.

Certain embodiments provide the use of a compound as described herein in the manufacture of a medicament for treating, preventing, or ameliorating obesity as described herein in a patient who is subsequently administered an additional agent or therapy as described herein.

Certain embodiments provide the use of a compound as described herein in the manufacture of a medicament for treating, preventing, or ameliorating diabetes as described herein in a patient who is subsequently administered an additional agent or therapy as described herein.

Certain embodiments provide the use of a compound as described herein in the manufacture of a medicament for treating, preventing, or ameliorating metabolic syndrome as described herein in a patient who is subsequently administered an additional agent or therapy as described herein.

Certain embodiments provide a kit for treating, preventing, or ameliorating metabolic disease as described herein wherein the kit comprises:
(i) a compound as described herein; and alternatively
(ii) an additional agent or therapy as described herein.

Certain embodiments provide a kit for treating, preventing, or ameliorating obesity as described herein wherein the kit comprises:
(i) a compound as described herein; and alternatively
(ii) an additional agent or therapy as described herein.

Certain embodiments provide a kit for treating, preventing, or ameliorating diabetes as described herein wherein the kit comprises:
(i) a compound as described herein; and alternatively
(ii) an additional agent or therapy as described herein.

Certain embodiments provide a kit for treating, preventing, or ameliorating metabolic syndrome as described herein wherein the kit comprises:
(i) a compound as described herein; and alternatively
(ii) an additional agent or therapy as described herein.

A kit as described herein may further include instructions for using the kit to treat, prevent, or ameliorate metabolic disease as described herein by combination therapy as described herein. In certain embodiments, the metabolic disease is obesity. In certain embodiments, the metabolic disease is diabetes.

### Antisense Compounds

Oligomeric compounds include, but are not limited to, oligonucleotides, oligonucleosides, oligonucleotide analogs, oligonucleotide mimetics, antisense compounds, antisense oligonucleotides, siRNAs and shRNAs. An oligomeric compound may be “antisense” to a target nucleic acid, meaning that is is capable of undergoing hybridization to a target nucleic acid through hydrogen bonding.

In certain embodiments, an antisense compound has a nucleobase sequence that, when written in the 5′ to 3′ direction, comprises the reverse complement of the target segment of a target nucleic acid to which it is targeted. In certain such embodiments, an antisense oligonucleotide has a nucleobase sequence that, when written in the 5′ to 3′ direction, comprises the reverse complement of the target segment of a target nucleic acid to which it is targeted.

In certain embodiments, an antisense compound targeted to a kallikrein nucleic acid is 12 to 30 subunits in length. In other words, such antisense compounds are from 12 to 30 linked subunits. In other embodiments, the antisense compound is 8 to 80, 12 to 50, 15 to 30, 18 to 24, 19 to 22, or 20 linked subunits. In certain such embodiments, the antisense compounds are 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, or 80 linked subunits in length, or a range defined by any two of the above values. In some embodiments the antisense compound is an antisense oligonucleotide, and the linked subunits are nucleosides.

In certain embodiments antisense oligonucleotides targeted to a kallikrein nucleic acid may be shortened or truncated. For example, a single subunit may be deleted from the 5′ end (5′ truncation), or alternatively from the 3′ end (3′ truncation). A shortened or truncated antisense compound targeted to a kallikrein nucleic acid may have two subunits deleted from the 5′ end, or alternatively may have two subunits deleted from the 3′ end, of the antisense compound. Alternatively, the deleted nucleosides may be dispersed throughout the antisense compound, for example, in an antisense compound having one nucleoside deleted from the 5′ end and one nucleoside deleted from the 3′ end.

When a single additional subunit is present in a lengthened antisense compound, the additional subunit may be located at the 5′ or 3′ end of the antisense compound. When two or more additional subunits are present, the added subunits may be adjacent to each other, for example, in an antisense compound having two subunits added to the 5′ end (5′ addition), or alternatively to the 3′ end (3′ addition), of the antisense compound. Alternatively, the added subunits may be dispersed throughout the antisense compound, for example, in an antisense compound having one subunit added to the 5′ end and one subunit added to the 3′ end.

It is possible to increase or decrease the length of an antisense compound, such as an antisense oligonucleotide, and/or introduce mismatch bases without eliminating activity. For example, in Woolf et al. (Proc. Natl. Acad. Sci. USA 89:7305-7309, 1992), a series of antisense oligonucleotides 13-25 nucleobases in length were tested for their ability to induce cleavage of a target RNA in an oocyte injection model. Antisense oligonucleotides 25 nucleobases in length with 8 or 11 mismatch bases near the ends of the antisense oligonucleotides were able to direct specific cleavage of the target mRNA, albeit to a lesser extent than the antisense oligonucleotides that contained no mismatches. Similarly, target specific cleavage was achieved using 13 nucleobase antisense oligonucleotides, including those with 1 or 3 mismatches.

Gautschi et al (J. Natl. Cancer Inst. 93;463-471, March 2001) demonstrated the ability of an oligonucleotide having 100% complementarity to the bcl-2 mRNA and having 3 mismatches to the bcl-xL mRNA to reduce the expression of both bcl-2 and bcl-xL in vitro and in vivo. Furthermore, this oligonucleotide demonstrated potent anti-tumor activity in vivo.

Maher and Dolnick (Nuc. Acid. Res. 16:3341-3358, 1988) tested a series of tandem 14 nucleobase antisense oligonucleotides, and a 28 and 42 nucleobase antisense oligonucleotides comprised of the sequence of two or three of the tandem antisense oligonucleotides, respectively, for their ability to arrest translation of human DHFR in a rabbit reticulocyte assay. Each of the three 14 nucleobase antisense oligonucleotides alone was able to inhibit translation, albeit at a more modest level than the 28 or 42 nucleobase antisense oligonucleotides.

### Antisense Compound Motifs

In certain embodiments, antisense compounds targeted to a kallikrein nucleic acid have chemically modified subunits arranged in patterns, or motifs, to confer to the antisense compounds properties such as enhanced inhibitory activity, increased binding affinity for a target nucleic acid, or resistance to degradation by in vivo nucleases.

Chimeric antisense compounds typically contain at least one region modified so as to confer increased resistance to nuclease degradation, increased cellular uptake, increased binding affinity for the target nucleic acid, and/or increased inhibitory activity. A second region of a chimeric antisense compound may optionally serve as a substrate for the cellular endonuclease RNase H, which cleaves the RNA strand of an RNA:DNA duplex.

Antisense compounds having a gapmer motif are considered chimeric antisense compounds. In a gapmer an internal region having a plurality of nucleotides that supports RNaseH cleavage is positioned between external regions having a plurality of nucleotides that are chemically distinct from the nucleosides of the internal region. In the case of an antisense oligonucleotide having a gapmer motif, the gap segment generally serves as the substrate for endonuclease cleavage, while the wing segments comprise modified nucleosides. In certain embodiments, the regions of a gapmer are differentiated by the types of sugar moieties comprising each distinct region. The types of sugar moieties that are used to differentiate the regions of a gapmer may in some embodiments include β-D-ribonucleosides, β-D-deoxyribonucleosides, 2′-modified nucleosides (such 2′-modified nucleosides may include 2′-MOE, and 2′-O—CH₃, among others), and bicyclic sugar modified nucleosides (such bicyclic sugar modified nucleosides may include those having a 4′-(CH2)n—O-2′ bridge, where n=1 or n=2). Preferably, each distinct region comprises uniform sugar moieties. The wing-gap-wing motif is frequently described as “X-Y-Z”, where “X” represents the length of the 5′ wing region, “Y” represents the length of the gap region, and “Z” represents the length of the 3′ wing region. As used herein, a gapmer described as “X-Y-Z” has a configuration such that the gap segment is positioned immediately adjacent to each of the 5′ wing segment and the 3′ wing segment. Thus, no intervening nucleotides exist between the 5′ wing segment and gap segment, or the gap segment and the 3′ wing segment. Any of the antisense compounds described herein can have a gapmer motif. In some embodiments, X and Z are the same, in other embodiments they are different. In a preferred embodiment, Y is between 8 and 15 nucleotides. X, Y or Z can be any of 1, 2, 3, 4, 5, 6, 7, 8, 9, 10, 11, 12, 13, 14, 15, 16, 17, 18, 19, 20, 25, 30 or more nucleotides. Thus, gapmers of the present invention include, but are not limited to, for example 5-10-5, 4-8-4, 4-12-3, 4-12-4, 3-14-3, 2-13-5, 2-16-2, 1-18-1, 3-10-3, 2-10-2, 1-10-1, 2-8-2, 5-8-5, or 6-8-6.

In certain embodiments, the antisense compound has a “wingmer” motif, having a wing-gap or gap-wing configuration, i.e. an X-Y or Y-Z configuration as described above for the gapmer configuration. Thus, wingmer configurations of the present invention include, but are not limited to, for example 5-10, 8-4, 4-12, 12-4, 3-14, 16-2, 18-1, 10-3, 2-10, 1-10, 8-2, 2-13, 5-13, 5-8, or 6-8.

In certain embodiments, antisense compounds targeted to a kallikrein nucleic acid possess a 5-10-5 gapmer motif.

In certain embodiments, antisense compounds targeted to a kallikrein nucleic acid possess a 3-14-3 gapmer motif.

In certain embodiments, antisense compounds targeted to a kallikrein nucleic acid possess a 2-13-5 gapmer motif.

In certain embodiments, antisense compounds targeted to a kallikrein nucleic acid possess a 5-8-5 gapmer motif.

In certain embodiments, antisense compounds targeted to a kallikrein nucleic acid possess a 6-8-6 gapmer motif.

In certain embodiments, an antisense compound targeted to a kallikrein nucleic acid has a gap-widened motif.

In certain embodiments, a gap-widened antisense oligonucleotide targeted to a kallikrein nucleic acid has a gap segment of fourteen 2′-deoxyribonucleotides positioned immediately adjacent to and between wing segments of three chemically modified nucleosides. In certain embodiments, the chemical modification comprises a 2′-sugar modification. In another embodiment, the chemical modification comprises a 2′-MOE sugar modification.

In certain embodiments, a gap-widened antisense oligonucleotide targeted to a kallikrein nucleic acid has a gap segment of thirteen 2′-deoxyribonucleotides positioned immediately adjacent to and between a 5′ wing segment of two chemically modified nucleosides and a 3′ wing segment of five chemically modified nucleosides. In certain embodiments, the chemical modification comprises a 2′-sugar modification. In another embodiment, the chemical modification comprises a 2′-MOE sugar modification.

### Target Nucleic Acids, Target Regions and Nucleotide Sequences

Nucleotide sequences that encode kallikrein include, without limitation, the following: GENBANK Accession No. NM_{—}000892.3 (incorporated herein as SEQ ID NO: 1), GENBANK Accession No. DC412984.1 (incorporated herein as SEQ ID NO: 2), GENBANK Accession No. CN265612.1 (incorporated herein as SEQ ID NO: 3), GENBANK Accession No. AK297672.1 (incorporated herein as SEQ ID NO: 4), GENBANK Accession No. DC413312.1 (incorporated herein as SEQ ID NO: 5), GENBANK Accession No. AV688858.2 (incorporated herein as SEQ ID NO: 6), GENBANK, Accession No. CD652077.1 (incorporated herein as SEQ ID NO: 7), GENBANK Accession No. BC143911.1 (incorporated herein as SEQ ID NO: 8), GENBANK Accession No. CB162532.1 (incorporated herein as SEQ ID NO: 9), GENBANK Accession No. NT_{—}016354.19 truncated from nucleobases 111693001 to 111730000 (incorporated herein as SEQ ID NO: 10), GENBANK Accession No. NM_{—}008455.2 (incorporated herein as SEQ ID NO: 11), GENBANK Accession No. BB598673.1 (incorporated herein as SEQ ID NO: 12), the complement of GENBANK Accession No. NT_{—}039460.7 truncated from nucleobases 6114001 to 6144000 (incorporated herein as SEQ ID NO: 13), GENBANK Accession No. NM_{—}012725.2 (incorporated herein as SEQ ID NO: 14), GENBANK Accession No. NW_{—}047473.1 truncated from nucleobases 10952001 to 10982000 (incorporated herein as SEQ ID NO: 15), exons 1-7 and 9-15 cut from the rhesus genomic sequence GENBANK Accession No. NW_{—}001118167.1 truncated from 2358000 to 2391000 (incorporated herein as SEQ ID NO: 16), GENBANK Accession No. XM_{—}002804276.1 (incorporated herein as SEQ ID NO: 17), GENBANK Accession No. NW_{—}001118167.1. truncated from nucleobases 2358000 to 2391000 (incorporated herein as SEQ ID NO: 18), and exons 1-15 of the baboon sequence assembled from trace archive based on homology to human (incorporated herein as SEQ ID NO: 19).

It is understood that the sequence set forth in each SEQ ID NO in the Examples contained herein is independent of any modification to a sugar moiety, an internucleoside linkage, or a nucleobase. As such, antisense compounds defined by a SEQ ID NO may comprise, independently, one or more modifications to a sugar moiety, an internucleoside linkage, or a nucleobase. Antisense compounds described by Isis Number (Isis No) indicate a combination of nucleobase sequence and motif.

In certain embodiments, a target region is a structurally defined region of the target nucleic acid. For example, a target region may encompass a 3′ UTR, a 5′ UTR, an exon, an intron, an exon/intron junction, a coding region, a translation initiation region, translation termination region, or other defined nucleic acid region. The structurally defined regions for kallikrein can be obtained by accession number from sequence databases such as NCBI and such information is incorporated herein by reference. In certain embodiments, a target region may encompass the sequence from a 5′ target site of one target segment within the target region to a 3′ target site of another target segment within the same target region.

Targeting includes determination of at least one target segment to which an antisense compound hybridizes, such that a desired effect occurs. In certain embodiments, the desired effect is a reduction in mRNA target nucleic acid levels. In certain embodiments, the desired effect is reduction of levels of protein encoded by the target nucleic acid or a phenotypic change associated with the target nucleic acid.

A target region may contain one or more target segments. Multiple target segments within a target region may be overlapping. Alternatively, they may be non-overlapping. In certain embodiments, target segments within a target region are separated by no more than about 300 nucleotides. In certain embodiments, target segments within a target region are separated by a number of nucleotides that is, is about, is no more than, is no more than about, 250, 200, 150, 100, 90, 80, 70, 60, 50, 40, 30, 20, or 10 nucleotides on the target nucleic acid, or is a range defined by any two of the proceeding values. In certain embodiments, target segments within a target region are separated by no more than, or no more than about, 5 nucleotides on the target nucleic acid. In certain embodiments, target segments are contiguous. Contemplated are target regions defined by a range having a starting nucleic acid that is any of the 5′ target sites or 3′ target sites listed herein.

Suitable target segments may be found within a 5′ UTR, a coding region, a 3′ UTR, an intron, an exon, or an exon/intron junction. Target segments containing a start codon or a stop codon are also suitable target segments. A suitable target segment may specifically exclude a certain structurally defined region such as the start codon or stop codon.

The determination of suitable target segments may include a comparison of the sequence of a target nucleic acid to other sequences throughout the genome. For example, the BLAST algorithm may be used to identify regions of similarity amongst different nucleic acids. This comparison can prevent the selection of antisense compound sequences that may hybridize is a non-specific manner to sequences other than a selected target nucleic acid (i.e., non-target or off-target sequences).

There may be variation in activity (e.g., as defined by percent reduction of target nucleic acid levels) of the antisense compounds within an active target region. In certain embodiments, reductions in kallikrein mRNA levels are indicative of inhibition of kallikrein expression. Reductions in levels of a kallikrein protein are also indicative of inhibition of target mRNA expression. Further, phenotypic changes are indicative of inhibition of kallikrein expression. For example, in certain embodiments, reduced body weight, reduced body fat content, reduced body fat depot, reduced blood glucose, reduced blood insulin, and/or reduced plasma triglycerides may be indicative of inhibition of kallikrein expression. In certain embodiments, increase glucose tolerance and/or increased insulin tolerance may be indicative of inhibition of kallikrein expression.

### Hybridization

In some embodiments, hybridization occurs between an antisense compound disclosed herein and a kallikrein nucleic acid. The most common mechanism of hybridization involves hydrogen bonding (e.g., Watson-Crick, Hoogsteen or reversed Hoogsteen hydrogen bonding) between complementary nucleobases of the nucleic acid molecules.

Hybridization can occur under varying conditions. Stringent conditions are sequence-dependent and are determined by the nature and composition of the nucleic acid molecules to be hybridized.

Methods of determining whether a sequence is specifically hybridizable to a target nucleic acid are well known in the art. In certain embodiments, the antisense compounds provided herein are specifically hybridizable with a kallikrein nucleic acid.

### Complementarity

An antisense compound and a target nucleic acid are complementary to each other when a sufficient number of nucleobases of the antisense compound can hydrogen bond with the corresponding nucleobases of the target nucleic acid, such that a desired effect will occur (e.g., antisense inhibition of a target nucleic acid, such as a kallikrein nucleic acid).

Non-complementary nucleobases between an antisense compound and a kallikrein nucleic acid may be tolerated provided that the antisense compound remains able to specifically hybridize to a target nucleic acid. Moreover, an antisense compound may hybridize over one or more segments of a kallikrein nucleic acid such that intervening or adjacent segments are not involved in the hybridization event (e.g., a loop structure, mismatch or hairpin structure).

In certain embodiments, the antisense compounds provided herein, or a specified portion thereof, are, or are at least, 70%, 80%, 85%, 86%, 87%, 88%, 89%, 90%, 91%, 92%, 93%, 94%, 95%, 96%, 97%, 98%, 99%, or 100% complementary to a kallikrein nucleic acid, a target region, target segment, or specified portion thereof. Percent complementarity of an antisense compound with a target nucleic acid can be determined using routine methods.

For example, an antisense compound in which 18 of 20 nucleobases of the antisense compound are complementary to a target region, and would therefore specifically hybridize, would represent 90 percent complementarity. In this example, the remaining noncomplementary nucleobases may be clustered or interspersed with complementary nucleobases and need not be contiguous to each other or to complementary nucleobases. As such, an antisense compound which is 18 nucleobases in length having 4 (four) noncomplementary nucleobases which are flanked by two regions of complete complementarity with the target nucleic acid would have 77.8% overall complementarity with the target nucleic acid and would thus fall within the scope of the present invention. Percent complementarity of an antisense compound with a region of a target nucleic acid can be determined routinely using BLAST programs (basic local alignment search tools) and PowerBLAST programs known in the art (Altschul et. al., J. Mol. Biol., 1990, 215, 403 410; Zhang and Madden, Genome Res., 1997, 7, 649 656). Percent homology, sequence identity or complementarity, can be determined by, for example, the Gap program (Wisconsin Sequence Analysis Package, Version 8 for Unix, Genetics Computer Group, University Research Park, Madison Wis.), using default settings, which uses the algorithm of Smith and Waterman (Adv. Appl. Math., 1981, 2, 482 489).

In certain embodiments, the antisense compounds provided herein, or specified portions thereof, are fully complementary (i.e., 100% complementary) to a target nucleic acid, or specified portion thereof. For example, an antisense compound may be fully complementary to a kallikrein nucleic acid, or a target region, or a target segment or target sequence thereof. As used herein, “fully complementary” means each nucleobase of an antisense compound is capable of precise base pairing with the corresponding nucleobases of a target nucleic acid. For example, a 20 nucleobase antisense compound is fully complementary to a target sequence that is 400 nucleobases long, so long as there is a corresponding 20 nucleobase portion of the target nucleic acid that is fully complementary to the antisense compound. Fully complementary can also be used in reference to a specified portion of the first and/or the second nucleic acid. For example, a 20 nucleobase portion of a 30 nucleobase antisense compound can be “fully complementary” to a target sequence that is 400 nucleobases long. The 20 nucleobase portion of the 30 nucleobase oligonucleotide is fully complementary to the target sequence if the target sequence has a corresponding 20 nucleobase portion wherein each nucleobase is complementary to the 20 nucleobase portion of the antisense compound. At the same time, the entire 30 nucleobase antisense compound may or may not be fully complementary to the target sequence, depending on whether the remaining 10 nucleobases of the antisense compound are also complementary to the target sequence.

The location of a non-complementary nucleobase may be at the 5′ end or 3′ end of the antisense compound. Alternatively, the non-complementary nucleobase or nucleobases may be at an internal position of the antisense compound. When two or more non-complementary nucleobases are present, they may be contiguous (i.e., linked) or non-contiguous. In one embodiment, a non-complementary nucleobase is located in the wing segment of a gapmer antisense oligonucleotide.

In certain embodiments, antisense compounds that are, or are up to 12, 13, 14, 15, 16, 17, 18, 19, or 20 nucleobases in length comprise no more than 4, no more than 3, no more than 2, or no more than 1 non-complementary nucleobase(s) relative to a target nucleic acid, such as a kallikrein nucleic acid, or specified portion thereof.

In certain embodiments, antisense compounds that are, or are up to 12, 13, 14, 15, 16, 17, 18, 19, 20, 21, 22, 23, 24, 25, 26, 27, 28, 29, or 30 nucleobases in length comprise no more than 6, no more than 5, no more than 4, no more than 3, no more than 2, or no more than 1 non-complementary nucleobase(s) relative to a target nucleic acid, such as a kallikrein nucleic acid, or specified portion thereof.

The antisense compounds provided herein also include those which are complementary to a portion of a target nucleic acid. As used herein, “portion” refers to a defined number of contiguous (i.e. linked) nucleobases within a region or segment of a target nucleic acid. A “portion” can also refer to a defined number of contiguous nucleobases of an antisense compound. In certain embodiments, the antisense compounds, are complementary to at least an 8 nucleobase portion of a target segment. In certain embodiments, the antisense compounds are complementary to at least a 12 nucleobase portion of a target segment. In certain embodiments, the antisense compounds are complementary to at least a 15 nucleobase portion of a target segment. Also contemplated are antisense compounds that are complementary to at least a 9, 10, 11, 12, 13, 14, 15, 16, 17, 18, 19, 20, or more nucleobase portion of a target segment, or a range defined by any two of these values.

### Identity

The antisense compounds provided herein may also have a defined percent identity to a particular nucleotide sequence, SEQ ID NO, or compound represented by a specific Isis number, or portion thereof. As used herein, art antisense compound is identical to the sequence disclosed herein if it has the same nucleobase pairing ability. For example, a RNA which contains uracil in place of thymidine in a disclosed DNA sequence would be considered identical to the DNA sequence since both uracil and thymidine pair with adenine. Shortened and lengthened versions of the antisense compounds described herein as well as compounds having non-identical bases relative to the antisense compounds provided herein also are contemplated. The non-identical bases may be adjacent to each other or dispersed throughout the antisense compound. Percent identity of an antisense compound is calculated according to the number of bases that have identical base pairing relative to the sequence to which it is being compared.

In certain embodiments, the antisense compounds, or portions thereof, are at least 70%, 75%, 80%, 85%, 90%, 95%, 96%, 97%, 98%, 99% or 100% identical to one or more of the antisense compounds or SEQ ID NOs, or a portion thereof, disclosed herein.

In certain embodiments, a portion of the antisense compound is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

In certain embodiments, a portion of the antisense oligonucleotide is compared to an equal length portion of the target nucleic acid. In certain embodiments, an 8, 9, 10, 11, 12, 13, 14, 15, 16, 17, 18, 19, 20, 21, 22, 23, 24, or 25 nucleobase portion is compared to an equal length portion of the target nucleic acid.

### Modifications

A nucleoside is a base-sugar combination. The nucleobase (also known as base) portion of the nucleoside is normally a heterocyclic base moiety. Nucleotides are nucleosides that further include a phosphate group covalently linked to the sugar portion of the nucleoside. For those nucleosides that include a pentofuranosyl sugar, the phosphate group can be linked to the 2′, 3′ or 5′ hydroxyl moiety of the sugar. Oligonucleotides are formed through the covalent linkage of adjacent nucleosides to one another, to form a linear polymeric oligonucleotide. Within the oligonucleotide structure, the phosphate groups are commonly referred to as forming the internucleoside linkages of the oligonucleotide.

Modifications to antisense compounds encompass substitutions or changes to internucleoside linkages, sugar moieties, or nucleobases. Modified antisense compounds are often preferred over native forms because of desirable properties such as, for example, enhanced cellular uptake, enhanced affinity for nucleic acid target, increased stability in the presence of nucleases, or increased inhibitory activity.

Chemically modified nucleosides may also be employed to increase the binding affinity of a shortened or truncated antisense oligonucleotide for its target nucleic acid. Consequently, comparable results can often be obtained with shorter antisense compounds that have such chemically modified nucleosides.

### Modified Internucleoside Linkages

The naturally occuring internucleoside linkage of RNA and DNA is a 3′ to 5′ phosphodiester linkage. Antisense compounds having one or more modified, i.e., non-naturally occurring, internucleoside linkages are often selected over antisense compounds having naturally occurring internucleoside linkages because of desirable properties such as, for example, enhanced cellular uptake, enhanced affinity for target nucleic acids, and increased stability in the presence of nucleases.

Oligonucleotides having modified internucleoside linkages include internucleoside linkages that retain a phosphorus atom as well as internucleoside linkages that do not have a phosphorus atom. Representative phosphorus containing internucleoside linkages include, but are not limited to, phosphodiesters, phosphotriesters, methylphosphonates, phosphoramidate, and phosphorothioates. Methods of preparation of phosphorous-containing and non-phosphorous-containing linkages are well known.

In certain embodiments, antisense compounds targeted to a kallikrein nucleic acid comprise one or more modified internucleoside linkages. In certain embodiments, the modified internucleoside linkages are phosphorothioate linkages. In certain embodiments, each internucleoside linkage of an antisense compound is a phosphorothioate internucleoside linkage.

### Modified Sugar Moieties

Antisense compounds can optionally contain one or more nucleosides wherein the sugar group has been modified. Such sugar modified nucleosides may impart enhanced nuclease stability, increased binding affinity, or some other beneficial biological property to the antisense compounds. In certain embodiments, nucleosides comprise chemically modified ribofuranose ring moieties. Examples of chemically modified ribofuranose rings include without limitation, addition of substitutent groups (including 5′ and 2′ substituent groups, bridging of non-geminal ring atoms to form bicyclic nucleic acids (BNA), replacement of the ribosyl ring oxygen atom with S, N(R), or C(R₁)(R₂) (R, R₁ and R₂ each independently H, C₁-C₁₂ alkyl or a protecting group) and combinations thereof. Examples of chemically modified sugars include 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US2005-0130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH₃, 2′-OCH₂CH₃, 2′-OCH₂CH₂F and 2′-O(CH₂)₂OCH₃ substituent groups. The substituent at the 2′ position can also be selected from allyl, amino, azido, thio, O-allyl, O—C₁-C₁₀ alkyl, OCF₃, OCH₂F, O(CH₂)₂SCH₃, O(CH₂)₂—O—N(Rₘ)(Rₙ), O—CH₂—C(═O)—N(Rₘ)(Rₙ), and O—CH₂—C(═O)—N(R₁)—(CH₂)₂—N(Rₘ)(Rₙ), where each R₁, Rₘ and Rₙis, independently, H or substituted or unsubstituted C₁-C₁₀ alkyl.

As used herein, “bicyclic nucleosides” refer to modified nucleosides comprising a bicyclic sugar moiety. Examples of bicyclic nucleosides include without limitation nucleosides comprising a bridge between the 4′ and the 2′ ribosyl ring atoms. In certain embodiments, antisense compounds provided herein include one or more bicyclic nucleosides comprising a 4′ to 2′ bridge. Examples of such 4′ to 2′ bridged bicyclic nucleosides, include but are not limited to one of the formulae: 4′-(CH₂)—O-2′ (LNA); 4′-(CH₂)—S-2′; 4′-(CH₂)₂—O-2′ (ENA); 4′-CH(CH₃)—O-2′ (also referred to as constrained ethyl or cEt) and 4′-CH(CH₂OCH₃)—O-2′ (and analogs thereof see U.S. Pat. No. 7,399,845, issued on Jul. 15, 2008); 4′-C(CH₃)(CH₃)—O-2′ (and analogs thereof see published International Application WO/2009/006478, published Jan. 8, 2009); 4′-CH₂—N(OCH₃)-2′ (and analogs thereof see published International Application WO/2008/150729, published Dec. 11, 2008); 4′-CH₂—O—N(CH₃)-2′ (see published U.S. Patent Application US2004-0171570, published Sep. 2, 2004); 4′-CH₂—N(R)—O-2′, wherein R is H, C₁-C₁₂ alkyl, or a protecting group (see U.S. Pat. No. 7,427,672, issued on Sep. 23, 2008); 4′-CH₂—C(H)(CH₃)-2′ (see Chattopadhyaya et. al., J. Org. Chem., 2009, 74, 118-134); and 4′-CH₂—C(═CH₂)-2′ (and analogs thereof see published International Application WO 2008/154401, published on Dec. 8, 2008).

Further reports related to bicyclic nucleosides can also be found is published literature (see for example: Singh et al., Chem. Commun., 1998, 4, 455-456; Koshkin et al., Tetrahedron, 1998, 54, 3607-3630; Wahlestedt et al., Proc. Natl. Acad. Sci. U.S.A., 2000, 97, 5633-5638; Kumar et al., Bioorg. Med. Chem. Lett., 1998, 8, 2219-2222; Singh et al., J. Org. Chem., 1998, 63, 10035-10039; Srivastava et al., J. Am. Chem. Soc., 2007, 129(26) 8362-8379; Elayadi et al., Curr. Opinion Invest. Drugs, 2001, 2, 558-561; Braasch et al., Chem. Biol., 2001, 8, 1-7; and Orum et al., Curr. Opinion Mol. Ther., 2001, 3, 239-243; U.S. Pat. Nos. 6,268,490; 6,525,191; 6,670,461; 6,770,748; 6,794,499; 7,034,133; 7,053,207; 7,399,845; 7,547,684; and 7,696,345; U.S. Patent Publication No. US2008-0039618; US2009-0012281; U.S. Patent Ser. Nos. 60/989,574; 61/026,995; 61/026,998; 61/056,564; 61/086,231; 61/097,787; and 61/099,844; Published PCT International applications WO 1994/014226; WO 2004/106356; WO 2005/021570; WO 2007/134181; WO 2008/150729; WO 2008/154401; and WO 2009/006478. Each of the foregoing bicyclic nucleosides can be prepared having one or more stereochemical sugar configurations including for example α-L-ribofuranose and β-D-ribofuranose (see PCT international application PCT/DK98/00393, published on Mar. 25, 1999 as WO 99/14226).

In certain embodiments, bicyclic sugar moieties of BNA nucleosides include, but are not limited to, compounds having at least one bridge between the 4′ and the 2′ position of the pentofuranosyl sugar moiety wherein such bridges independently comprises 1 or from 2 to 4 linked groups independently selected from —[C(Rₐ)(R_{b})]ₙ—, —C(Rₐ)═C(R_{b})—, —C(Rₐ)═N—, —C(═O)—, —C(═NRₐ)—, —C(═S)—, —O—, —Si(Rₐ)₂—, —S(═O)ₓ—, and —N(Rₐ)—;

wherein:

x is 0, 1, or 2;

n is 1, 2, 3, or 4;

each Rₐ and R_{b} is, independently, H, a protecting group, hydroxyl, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, heterocycle radical, substituted heterocycle radical, heteroaryl, substituted heteroaryl, C₅-C₇ alicyclic radical, substituted C₅-C₇ alicyclic radical, halogen, OJ₁, NJ₁J₂, SJ₁, N₃, COOJ₁, acyl (C(═O)—H, substituted acyl, CN, sulfonyl (S(═O)₂-J₁), or sulfoxyl (S(═O)-J₁); and

each J₁ and J₂ is, independently, H, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₅-C₂₀ aryl, substituted C₅-C₂₀ aryl, acyl (C(═O)—H, substituted acyl, a heterocycle radical, a substituted heterocycle radical, C₁-C₁₂ aminoalkyl, substituted C₁-C₁₂ aminoalkyl or a protecting group.

In certain embodiments, the bridge of a bicyclic sugar moiety is —[C(Rₐ)(R_{b})]ₙ—, —[C(Rₐ)(R_{b})]ₐ—O—, —C(RₐR_{b})—N(R)—O— or —C(RₐR_{b})—O—N(R)—. In certain embodiments, the bridge is 4′-CH₂-2′, 4′-(CH₂)₂-2′, 4′-(CH₂)₃-2′, 4′-CH₂—O-2′, 4′-(CH₂)₂—O-2′, 4′-CH₂O—N(R)-2′ and 4′-CH₂—N(R)—O-2′- wherein each R is, independently, H, a protecting group of C₁-C₁₂ alkyl.

In certain embodiments, bicyclic nucleosides are further defined by isomeric configuration. For example, a nucleoside comprising a 4′-2′ methylene-oxy bridge, may be in the α-L configuration or in the β-D configuration. Previously, α-L-methyleneoxy (4′-CH₂—O-2′) BNA's have been incorporated into antisense oligonucleotides that showed antisense activity (Frieden et al., Nucleic Acids Research, 2003, 21, 6365-6372).

In certain embodiments, bicyclic nucleosides includes, but are not limited to, (A) α-L-methyleneoxy (4′-CH₂—O-2′) BNA, (B) β-D-methyleneoxy (4′-CH₂—O-2′) BNA, (C) ethyleneoxy (4′-(CH₂)₂—O-2′) BNA, (D) aminooxy (4′-CH₂—O—N(R)-2′) BNA, (E) oxyamino (4′-CH₂—N(R)—O-2′) BNA, and (F) methyl(methyleneoxy) (4′-CH(CH₃)—O-2′) BNA, (G) methylene-thio (4′-CH₂—S-2′)BNA, (H) methylene-amino (4′-CH₂—N(R)-2′) BNA, (I) methyl carbocyclic (4′-CH₂—CH(CH₃)-2′) BNA, (J) propylene carbocyclic (4′-(CH₂)₃-2′) BNA and (K) vinyl BNA as depicted below.

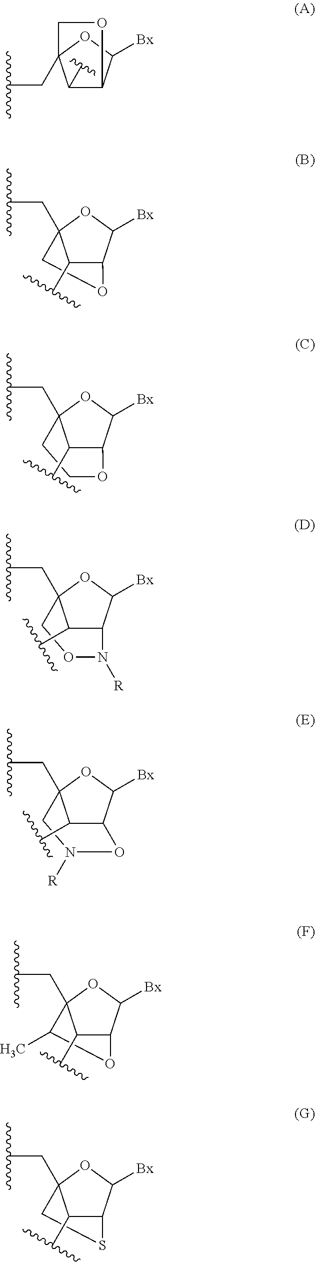

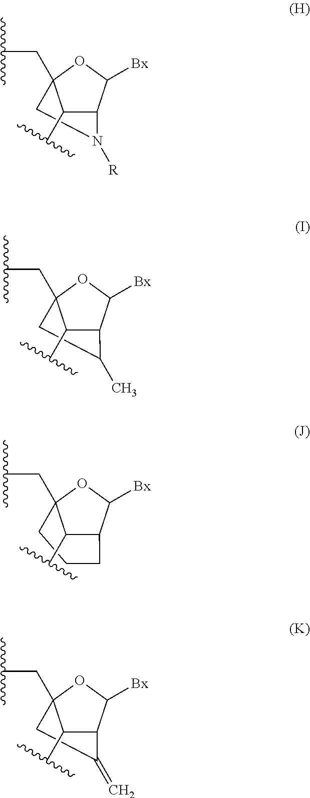

wherein Bx is the base moiety and R is independently H, a protecting group, C₁-C₁₂ alkyl or C₁-C₁₂ alkoxy.

In certain embodiments, bicyclic nucleosides are provided having Formula I:

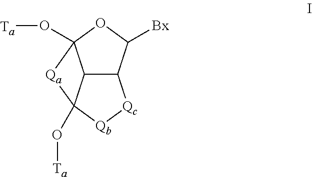

wherein:

Bx is a heterocyclic base moiety;

-Qₐ-Q_{b}-Q_{c}- is —CH₂—N(R_{c})—CH₂—, —C(═O)—N(R_{c})—CH₂—, —CH₂—O—N(R_{c})—, —CH₂—N(R_{c})—O— or —N(R_{c})—O—CH₂;

R_{c} is C₁-C₁₂ alkyl or an amino protecting group; and

Tₐ and T_{b} are each, independently H, a hydroxyl protecting group, a conjugate group, a reactive phosphorus group, a phosphorus moiety or a covalent attachment to a support medium.

In certain embodiments, bicyclic nucleosides are provided having Formula II:

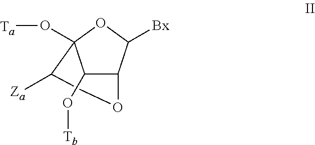

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Zₐ is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl, acyl, substituted acyl, substituted amide, thiol or substituted thio.

In one embodiment, each of the substituted groups is, independently, mono or poly substituted with substituent groups independently selected from halogen, oxo, hydroxyl, OJ_{c}, NJ_{c}J_{d}, SJ_{c}, N₃, OC(═X)J_{c}, and NJ_{c}C(═X)NJ_{c}J_{d}, wherein each J_{c}, J_{d} and Jₑ is, independently, H, C₁-C₆ alkyl, or substituted C₁-C₆ alkyl and X is O or NJ_{c}.

In certain embodiments, bicyclic nucleosides are provided having Formula III:

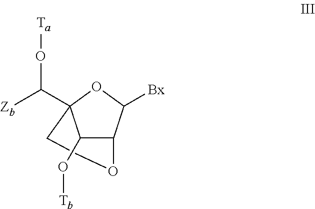

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

Z_{b} is C₁-C₆ alkyl, C₂-C₆ alkenyl, C₂-C₆ alkynyl, substituted C₁-C₆ alkyl, substituted C₂-C₆ alkenyl, substituted C₂-C₆ alkynyl or substituted acyl (C(═O)—).

In certain embodiments, bicyclic nucleosides are provided having Formula IV:

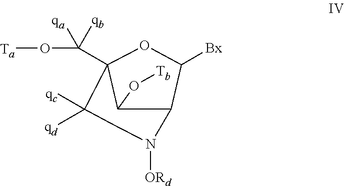

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

R_{d} is C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl;

each qₐ, q_{b}, q_{c} and q_{d} is, independently, H, halogen, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl, C₁-C₆ alkoxy, substituted C₁-C₆ alkoxyl, acyl, substituted acyl, C₁-C₆ aminoalkyl or substituted C₁-C₆ aminoalkyl;

In certain embodiments, bicyclic nucleosides are provided having Formula V:

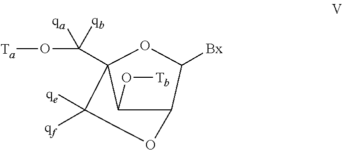

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

qₐ, q_{b}, qₑ and q_{f} are each, independently, hydrogen, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxy, substituted C₁-C₁₂ alkoxy, OJⱼ, SJⱼ, SOJⱼ, SO₂Jⱼ, NJⱼJₖ, Nⱼ, CN, C(═O)OJⱼ, C(═O)NJⱼJₖ, C(═)Jⱼ, O—C(═O)NJⱼJₖ, N(H)C(═NH)NJⱼJₖ, N(H)C(═O)NJⱼJₖ or N(H)C(═S)NJⱼJₖ;

or qₑ and q_{f} together are ═C(q_{g})(qₕ);

q_{g} and qₕ are each, independently, H, halogen, C₁-C₁₂ alkyl or substituted C₁-C₁₂ alkyl.

The synthesis and preparation of the methyleneoxy (4′-CH₂—O-2′) BNA monomers adenine, cytosine, guanine, 5-methyl-cytosine, thymine and uracil, along with their oligomerization, and nucleic acid recognition properties have been described (Koshkin et al., Tetrahedron, 1998, 54, 3607-3630). BNAs and preparation thereof are also described in WO 98/39352 and WO 99/14226.

Analogs of methyleneoxy (4′-CH₂—O-2′) BNA and 2′-thio-BNAs, have also been prepared (Kumar et al., Bioorg. Med. Chem. Lett., 1998, 8, 2219-2222). Preparation of locked nucleoside analogs comprising oligodeoxyribonucleotide duplexes as substrates for nucleic acid polymerases has also been described (Wengel et al., WO 99/14226). Furthermore, synthesis of 2′-amino-BNA, a novel comformationally restricted high-affinity oligonucleotide analog has been described in the art (Singh et al., J. Org. Chem., 1998, 63, 10035-10039). In addition, 2′-amino- and 2′-methylamino-BNA's have been prepared and the thermal stability of their duplexes with complementary RNA and DNA strands has been previously reported.

In certain embodiments, bicyclic nucleosides are provided having Formula VI:

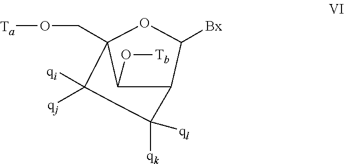

wherein:

Bx is a heterocyclic base moiety;

Tₐ and T_{b} are each, independently H, a hydroxyl protecting group, a conjugate group, a reactive phosphorus group, a phosphorus moiety or a covalent attachment to a support medium;

each qᵢ, qⱼ, qₖ and qₗ is, independently, H, halogen, C₁-C₁₂ alkyl, substituted C₁-C₁₂ alkyl, C₂-C₁₂ alkenyl, substituted C₂-C₁₂ alkenyl, C₂-C₁₂ alkynyl, substituted C₂-C₁₂ alkynyl, C₁-C₁₂ alkoxyl, substituted C₁-C₁₂ alkoxyl, OJⱼ, SJⱼ, SOJⱼ, SO₂Jⱼ, NJⱼJₖ, N₃, CN, C(═O)OJⱼ, C(═O)NJⱼJₖ, C(═O)Jⱼ, O—C(═O)NJⱼJₖ, N(H)C(═NH)NJⱼJₖ, N(H)C(═O)NJⱼJₖ or N(H)C(═S)NJⱼJₖ; and

qᵢ and qⱼ or qₗ and qₖ together are ═C(q_{g})(qₕ), wherein q_{g} and qₕ are each, independently, H, halogen, C₁-C₁₂ alkyl or substituted C₁-C₁₂ alkyl.

One carbocyclic bicyclic nucleoside having a 4′-(CH₂)₃-2′ bridge and the alkenyl analog bridge 4′-CH═CH—CH₂-2′ have been described (Freier et al., Nucleic Acids Research, 1997, 25(22), 4429-4443 and Albaek et al., J. Org. Chem., 2006, 71, 7731-7740). The synthesis and preparation of carbocyclic bicyclic nucleosides along with their oligomerization and biochemical studies have also been described (Srivastava et al., J. Am. Chem. Soc. 2007, 129(26), 8362-8379).

As used herein, “4′-2′ bicyclic nucleoside” or “4′ to 2′ bicyclic nucleoside” refers to a bicyclic nucleoside comprising a furanose ring comprising a bridge connecting two carbon atoms of the furanose ring connects the 2′ carbon atom and the 4′ carbon atom of the sugar ring.

As used herein, “monocylic nucleosides” refer to nucleosides comprising modified sugar moieties that are not bicyclic sugar moieties. In certain embodiments, the sugar moiety, or sugar moiety analogue, of a nucleoside may be modified or substituted at any position.

As used herein, “2′-modified sugar” means a furanosyl sugar modified at the 2′ position. In certain embodiments, such modifications include substituents selected from: a halide, including, but not limited to substituted and unsubstituted alkoxy, substituted and unsubstituted thioalkyl, substituted and unsubstituted amino alkyl, substituted and unsubstituted alkyl, substituted and unsubstituted allyl, and substituted and unsubstituted alkynyl. In certain embodiments, 2′ modifications are selected from substituents including, but not limited to: O[(CH₂)ₙO]ₘCH₃, O(CH₂)ₙNH₂, O(CH₂)ₙCH₃, O(CH₂)ₙF, O(CH₂)ₙONH₂, OCH₂C(═O)N(H)CH₂, and O(CH₂)ₙON[(CH₂)ₙCH₃]₂, where n and m are from 1 to 10. Other 2′-substituent groups can also be selected from: C₁-C₁₂ alkyl, substituted alkyl, alkenyl, alkynyl, alkaryl, aralkyl, O-alkaryl or O-aralkyl, SH, SCH₃, OCN, Cl, Br, CN, F, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, and RNA cleaving group, a reporter group, an intercalator, a group for improving pharmacokinetic properties, or a group for improving the pharmacodynamic properties of an antisense compound, and other substituents having similar properties. In certain embodiments, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, Helv. Chim. Acta, 1995, 78, 485-504; Altmann et al., Chimia, 1996, 50, 168-176; Altmann et al., Biochem. Soc. Trans., 1996, 24, 630-637; and Altmann et al., Nucleosides Nucleotides, 1997, 16, 917-926).

As used herein, a “modified tetrahydropyran nucleoside” or “modified THP nucleoside” means a nucleoside having a six-membered tetrahydropyran “sugar” substituted in for the pentofuranosyl residue in normal nucleosides (a sugar surrogate). Modified THP nucleosides include, but are not limited to, what is referred to in the art as hexitol nucleic acid (HNA), anitol nucleic acid (ANA), manitol nucleic acid (MNA) (see Leumann, Bioorg. Med. Chem., 2002, 10, 841-854) or fluoro HNA (F-HNA) having a tetrahydropyran ring system as illustrated below:

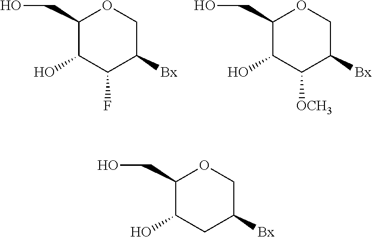

In certain embodiments, sugar surrogates are selected having Formula VII:

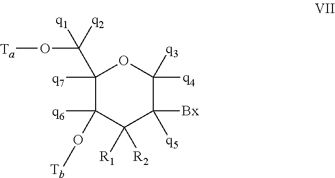

wherein independently for each of said at least one tetrahydropyran nucleoside analog of Formula VII:

Bx is a heterocyclic base moiety;

Tₐ and T_{b} are each, independently, an internucleoside linking group linking the tetrahydropyran nucleoside analog to the antisense compound or one of Tₐ and T_{b} is an internucleoside linking group linking the tetrahydropyran nucleoside analog to the antisense compound and the other of Tₐ and T_{b} is H, a hydroxyl protecting group, a linked conjugate group or a 5′ or 3′-terminal group;

q₁, q₂, q₃, q₄, q₅, q₆ and q₇ are each independently, H, C₁-C₆ alkyl, substituted C₁-C₆ alkyl, C₂-C₆ alkenyl, substituted C₂-C₆ alkenyl, C₂-C₆ alkynyl or substituted C₂-C₆ alkynyl; and each of R₁ and R₂ is selected from hydrogen, hydroxyl, halogen, subsitituted or unsubstituted alkoxy, NJ₁J₂, SJ₁, N₃, OC(═X)J₁, OC(═X)NJ₁J₂, NJ₃C(═X)NJ₁J₂ and CN, wherein X is O, S or NJ₁ and each J₁, J₂ and J₃ is, independently, H or C₁-C₆ alkyl.

In certain embodiments, the modified THP nucleosides of Formula VII are provided wherein q₁, q₂, q₃, q₄, q₅, q₆ and q₇ are each H. In certain embodiments, at least one q₁, q₂, q₃, q₄, q₅, q₆ and q₇ is other than H. In certain embodiments, at least one of q₁, q₂, q₃, q₄, q₅, q₆ and q₇ is methyl. In certain embodiments, THP nucleosides of Formula VII are provided wherein one of R₁ and R₂ is fluoro. In certain embodiments, R₁ is fluoro and R₂ is H; R₁ is methoxy and R₂ is H, and R₁ is methoxyethoxy and R₂ is H.

In certain embodiments, sugar surrogates comprise rings having more than 5 atoms and more than one heteroatom. For example nucleosides comprising morpholino sugar moieties and their use in oligomeric compounds has been reported (see for example: Braasch et al., Biochemistry, 2002, 41, 4503-4510; and U.S. Pat. Nos. 5,698,685; 5,166,315; 5,185,444; and 5,034,506). As used here, the term “morpholino” means a sugar surrogate having the following formula:

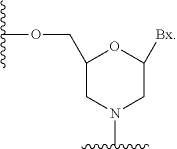

In certain embodiments, morpholinos may be modified, for example by adding or altering various substituent groups from the above morpholino structure. Such sugar surrogates are referred to herein as “modified morpholinos.”

Combinations of modifications are also provided without limitation, such as 2′-F-5′-methyl substituted nucleosides (see PCT International Application WO 2008/101157 published on Aug. 21, 2008 for other disclosed 5′, 2′-bis substituted nucleosides) and replacement of the ribosyl ring oxygen atom with S and further substitution at the 2′-position (see published U.S. Patent Application US2005-0138923, published on Jun. 16, 2005) or alternatively 5′-substitution of a bicyclic nucleic acid (see PCT International Application WO 2007/134181, published on Nov. 22, 2007 wherein a 4′-CH₂—O-2′ bicyclic nucleoside is further substituted at the 5′ position with a 5′-methyl or a 5′-vinyl group). The synthesis and preparation of carbocyclic bicyclic nucleosides along with their oligomerization and biochemical studies have also been described (see, e.g., Srivastava et al., J. Am. Chem. Soc., 2007, 129(26), 8362-8379).

In certain embodiments, antisense compounds comprise one or more modified, cyclohexenyl nucleosides, which is a nucleoside having a six-membered cyclohexenyl in place of the pentofuranosyl residue in naturally occurring nucleosides. Modified cyclohexenyl nucleosides include, but are not limited to those described in the art (see for example commonly owned, published PCT Application WO 2010/036696, published on Apr. 10, 2010, Robeyns et al., J. Am. Chem. Soc. 2008, 130(6), 1979-1984; Horvath et al., Tetrahedron Letters, 2007, 48, 3621-3623; Nauwelaerts et al., J. Am. Chem. Soc., 2007, 129(30), 9340-9348; Gu et al., Nucleosides, Nucleotides & Nucleic Acids, 2005, 24(5-7), 993-908; Nauwelaerts et al., Nucleic Acids Research, 2005, 33(8), 2452-2463; Robeyns et al., Acta Crystallographica, Section F: Structural Biology and Crystallization Communications, 2005, F61(6), 585-586; Gu et al., Tetrahedron, 2004, 60(9), 2111-2123; Gu et al., Oligonucleotides, 2003, 13(6), 479-489; Wang et al., J. Org. Chem., 2003, 68, 4499-4505; Verbeure et al., Nucleic Acids Research, 2001, 29(24), 4941-4947; Wang et al., J. Org. Chem., 2001, 66, 8478-82; Wang et al., Nucleosides, Nucleotides & Nucleic Acids, 2001, 20(4-7), 785-788; Wang et al., J. Am. Chem., 2000, 122, 8595-8602; Published PCI application, WO 06/047842; and Published PCT Application WO 01/049687; the text of each is incorporated by reference herein, in their entirety). Certain modified cyclohexenyl nucleosides have Formula X.

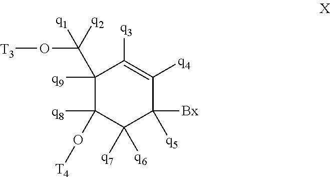

wherein independently for each of said at least one cyclohexenyl nucleoside analog of Formula X;

Bx is a heterocyclic base moiety;

T₃ and T₄ are each, independently, as internucleoside linking group linking the cyclohexenyl nucleoside analog to an antisense compound or one of T₃ and T₄ is an internucleoside linking group linking the tetrahydropyran nucleoside analog to an antisense compound and the other of T₃ and T₄ is H, a hydroxyl protecting group, a linked conjugate group, or a 5′- or 3′-terminal group; and

q₁, q₂, q₃, q₄, q₅, q₆, q₇, q₈ and q₉ are each, independently, H, C₁-C₆ alkyl, substituted C₁-C₆ alkyl, C₂-C₆ alkenyl, substituted C₂-C₆ alkenyl, C₂-C₆ alkynyl, substituted C₂-C₆ alkynyl or other sugar substituent group.

As used herein, “2′-modified” or “2′-substituted” refers to a nucleoside comprising a sugar comprising a substituent at the 2′ position other than H or OH. 2′-modified nucleosides, include, but are not limited to, bicyclic nucleosides wherein the bridge connecting two carbon atoms of the sugar ring connects the 2′ carbon and another carbon of the sugar ring; and nucleosides with non-bridging 2′substituents, such as allyl, amino, azido, thio, O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ and Rₙ is, independently, H or substituted or unsubstituted C₁-C₁₀ alkyl. 2′-modifed nucleosides may further comprise other modifications, for example at other positions of the sugar and/or at the nucleobase.

As used herein, “2′-F” refers to a nucleoside comprising a sugar comprising a fluoro group at the 2′ position of the sugar ring.

As used herein, “2′-OMe” or “2′-OCH₃” or “2′-O-methyl” each refers to a nucleoside comprising a sugar comprising an —OCH₃ group at the 2′ position of the sugar ring.

As used herein, “MOE” or “2′-MOE” or “2′-OCH₂CH₂OCH₃” or “2′-O-methoxyethyl” each refers to a nucleoside comprising a sugar comprising a —OCH₂CH₂OCH₃ group at the 2′ position of the sugar ring.

As used herein, “oligonucleotide” refers to a compound comprising a plurality of linked nucleosides. In certain embodiments, one or more of the plurality of nucleosides is modified. In certain embodiments, an oligonucleotide comprises one or more ribonucleosides (RNA) and/or deoxyribonucleosides (DNA).

Many other bicyclo and tricyclo sugar surrogate ring systems are also known in the art that can be used to modify nucleosides for incorporation into antisense compounds (see for example review article: Leumann, Bioorg. Med. Chem., 2002, 10, 841-854). Such ring systems can undergo various additional substitutions to enhance activity.

Methods for the preparations of modified sugars are well known to those skilled in the art. Some representative U.S. patents that teach the preparation of such modified sugars include without limitation, U.S. Pat. Nos. 4,981,957; 5,118,800; 5,319,080; 5,359,044; 5,393,878; 5,446,137; 5,466,786; 5,514,785; 5,519,134; 5,567,811; 5,576,427; 5,591,722; 5,597,909; 5,610,300; 5,627,053; 5,639,873; 5,646,265; 5,670,633; 5,700,920; 5,792,847 and 6,600,032 and International Application PCT/US2005/019219, filed Jun. 2, 2005 and published as WO 2005/121371 on Dec. 22, 2005, and each of which is herein incorporated by reference in its entirety.

In nucleotides having modified sugar moieties, the nucleobase moieties (natural, modified or a combination thereof) are maintained for hybridization with an appropriate nucleic acid target.

In certain embodiments, antisense compounds comprise one or more nucleosides having modified sugar moieties. In certain embodiments, the modified sugar moiety is 2′-MOE. In certain embodiments, the 2′-MOE modified nucleosides are arranged in a gapmer motif. In certain embodiments, the modified sugar moiety is a bicyclic nucleoside having a (4′-CH(CH₃)—O-2′) bridging group. In certain embodiments, the (4′-CH(CH₃)—O-2′) modified nucleosides are arranged throughout the wings of a gapmer motif.

### Compositions and Methods for Formulating Pharmaceutical Compositions

Antisense oligonucleotides may be admixed with pharmaceutically acceptable active or inert substances for the preparation of pharmaceutical compositions or formulations. Compositions and methods for the formulation of pharmaceutical compositions are dependent upon a number of criteria, including, but not limited to, route of administration, extent of disease, or dose to be administered.

An antisense compound targeted to a kallikrein nucleic acid can be utilized in pharmaceutical compositions by combining the antisense compound with a suitable pharmaceutically acceptable diluent or carrier. A pharmaceutically acceptable diluent includes phosphate-buffered saline (PBS). PBS is a diluent suitable for use in compositions to be delivered parenterally. Accordingly, its one embodiment, employed in the methods described herein is a pharmaceutical composition comprising an antisense compound targeted to a kallikrein nucleic acid and a pharmaceutically acceptable diluent. In certain embodiments, the pharmaceutically acceptable diluent is PBS. In certain embodiments, the antisense compound is an antisense oligonucleotide.

Pharmaceutical compositions comprising antisense compounds encompass any pharmaceutically acceptable salts, esters, or salts of such esters, or any other oligonucleotide which, upon administration to an animal, including a human, is capable of providing (directly or indirectly) the biologically active metabolite or residue thereof. Accordingly, for example, the disclosure is also drawn to pharmaceutically acceptable salts of antisense compounds, prodrugs, pharmaceutically acceptable salts of such prodrugs, and other bioequivalents. Suitable pharmaceutically acceptable salts include, but are not limited to, sodium and potassium salts.

A prodrug can include the incorporation of additional nucleosides at one or both ends of an antisense compound which are cleaved by endogenous nucleases within the body, to form the active antisense compound.

### Conjugated Antisense Compounds

Antisense compounds may be covalently linked to one or more moieties or conjugates which enhance the activity, cellular distribution or cellular uptake of the resulting antisense oligonucleotides. Typical conjugate groups include cholesterol moieties and lipid moieties. Additional conjugate groups include carbohydrates, phospholipids, biotin, phenazine, folate, phenanthridine, anthraquinone, acridine, fluoresceins, rhodamines, coumarins, and dyes.

Antisense compounds can also be modified to have one or more stabilizing groups that are generally attached to one or both termini of antisense compounds to enhance properties such as, for example, nuclease stability. Included in stabilizing groups are cap structures. These terminal modifications protect the antisense compound having terminal nucleic acid from exonuclease degradation, and can help in delivery and/or localization within a cell. The cap can be present at the 5′-terminus (5′-cap), or at the 3′-terminus (3′-cap), or can be present on both termini. Cap structures are well known in the art and include, for example, inverted deoxy abasic caps. Further 3′ and 5′-stabilizing groups that can be used to cap one or both ends of an antisense compound to impart nuclease stability include those disclosed in WO 03/004602 published on Jan. 16, 2003.

### Cell Culture and Antisense Compounds Treatment

The effects of antisense compounds on the level, activity or expression of kallikrein nucleic acids can be tested in vitro in a variety of cell types. Cell types used for such analyses are available from commerical vendors (e.g. American Type Culture Collection, Manassus, Va.; Zen-Bio, Inc., Research Triangle Park, N.C.; Clonetics Corporation, Walkersville, Md.) and are cultured according to the vendor's instructions using commercially available reagents (e.g. Invitrogen Life Technologies, Carlsbad, Calif.). Illustrative cell types include, but are not limited to, HepG2 cells, Hep3B cells, and primary hepatocytes.

### In Vitro Testing of Antisense Oligonucleotides

Described herein are methods for treatment of cells with antisense oligonucleotides, which can be modified appropriately for treatment with other antisense compounds.

In general, cells are treated with antisense oligonucleotides when the cells reach approximately 60-80% confluency in culture.

One reagent commonly used to introduce antisense oligonucleotides into cultured cells includes the cationic lipid transaction reagent LIPOFECTIN (Invitrogen, Carlsbad, Calif.). Antisense oligonucleotides are mixed with LIPOFECTIN in OPTI-MEM 1 (Invitrogen, Carlsbad, Calif.) to achieve the desired final concentration of antisense oligonucleotide and a LIPOFECTIN concentration that typically ranges 2 to 12 ug/mL per 100 nM antisense oligonucleotide.

Another reagent used to introduce antisense oligonucleotides into catered cells includes LIPOFECTAMINE (Invitrogen, Carlsbad, Calif.). Antisense oligonucleotide is mixed with LIPOFECTAMINE in OPTI-MEM 1 reduced serum medium (Invitrogen, Carlsbad, Calif.) to achieve the desired concentration of antisense oligonucleotide and a LIPOFECTAMINE concentration that typically ranges 2 to 12 ug/mL per 100 nM antisense oligonucleotide.

Another technique used to introduce antisense oligonucleotides into cultured cells includes electroporation.

Cells are treated with antisense oligonucleotides by routine methods. Cells are typically harvested 16-24 hours after antisense oligonucleotide treatment, at which time RNA or protein levels of target nucleic acids are measured by methods known in the art and described herein. In general, when treatments are performed in multiple replicates, the data are presented as the average of the replicate treatments.

The concentration of antisense oligonucleotide used varies from cell line to cell line. Methods to determine the optimal antisense oligonucleotide concentration for a particular cell line are well known in the art. Antisense oligonucleotides are typically used at concentrations ranging from 1 nM to 300 nM when transfected with LIPOFECTAMINE. Antisense oligonucleotides are used at higher concentrations ranging from 625 to 20,000 nM when transfected using electroporation.

### RNA Isolation

RNA analysis can be performed on total cellular RNA or poly(A)+mRNA. Methods of RNA isolation are well known in the art. RNA is prepared using methods well known in the art, for example, using the TRIZOL Reagent (Invitrogen, Carlsbad, Calif.) according to the manufacturer's recommended protocols.

### Analysis of Inhibition of Target Levels or Expression

Inhibition of levels or expression of a kallikrein nucleic acid can be assayed in a variety of ways known in the art. For example, target nucleic acid levels can be quantitated by, e.g., Northern blot analysis, competitive polymerase chain reaction (PCR), or quantitaive real-time PCR. RNA analysis can be performed on total cellular RNA or poly(A)+mRNA. Methods of RNA isolation are well known in the art. Northern blot analysis is also routine in the art. Quantitative real-time PCR can be conveniently accomplished using the commercially available ABI PRISM 7600, 7700, or 7900 Sequence Detection System, available from PE-Applied Biosystems, Foster City, Calif. and used according to manufacturer's instructions.

### Quantitative Real-Time PCR Analysis of Target RNA Levels

Quantitation of target RNA levels may be accomplished by quantitative real-time PCR using the ABI PRISM 7600, 7700, or 7900 Sequence Detection System (PE-Applied Biosystems, Foster City, Calif.) according to manufacturer's instructions. Methods of quantitative real-time PCR are well known in the art.

Prior to real-time PCR, the isolated RNA is subjected to a reverse transcriptase (RT) reaction, which produces complementary DNA (cDNA) that is then used as the substrate for the real-time PCR amplification. The RT and real-time PCR reactions are performed sequentially in the same sample well. RT and real-time PCR reagents are obtained from Invitrogen (Carlsbad, Calif.). RT real-time-PCR reactions are carried out by methods well known to those skilled in the art.

Gene (or RNA) target quantities obtained by real time PCR are normalized using either the expression level of a gene whose expression is constant, such as cyclophilin A, or by quantifying total RNA using RIBOGREEN (Invitrogen, Inc. Carlsbad, Calif.). Cyclophilin A expression is quantified by real time PCR, by being run simultaneously with the target, multiplexing, or separately. Total RNA is quantified using RIBOGREEN RNA quantification reagent (Invetrogen, Inc. Eugene, Oreg.), Methods of RNA quantification by RIBOGREEN are taught in Jones, L. J., et al, (Analytical Biochemistry, 1998, 265, 368-374). A CYTOFLUOR 4000 instrument (PE Applied Biosystems) is used to measure RIBOGREEN fluorescence.

Probes and primers are designed to hybridize to a kallikrein nucleic acid. Methods for designing real-time PCR probes and primers are well known in the art, and may include the use of software such as PRIMER EXPRESS Software (Applied Biosystems, Foster City, Calif.).

### Analysis of Protein Levels

Antisense inhibition of kallikrein nucleic acids can be assessed by measuring kallikrein protein levels. Protein levels of kallikrein can be evaluated or quantitated in a variety of ways well known in the art, such as immunoprecipitation, Western blot analysis (immunoblotting), enzyme-linked immunosorbent assay (ELISA), quantitative protein assays, protein activity assays (for example, caspase activity assays), immunohistochemistry, immunocytochemistry or fluorescence-activated cell sorting (FACS). Antibodies directed to a target can be identified and obtained to a variety of sources, such as the MSRS catalog of antibodies (Aerie Corporation, Birmingham, Mich.), or can be prepared via conventional monoclonal or polyclonal antibody generation methods well known in the art. Antibodies useful for the detection of mouse, rat, monkey, and human kallikrein are commercially available.

### In Vivo Testing of Antisense Compounds

Antisense compounds, for example, antisense oligonucleotides, are tested in animals to assess their ability to inhibit expression of kallikrein and produce phenotypic changes, such as, reduced body weight, reduced body fat content, reduced body fat depot, reduced blood glucose, reduced blood insulin, reduced plasma triglycerides, increased glucose tolerance, and/or increased insulin tolerance. Testing may be performed in normal animals, or in experimental disease models. For administration to animals, antisense oligonucleotides are formulated in a pharmaceutically acceptable diluent, such as phosphate-buffered saline. Administration includes parenteral routes of administration, such as intraperitoneal, intravenous, and subcutaneous. Calculation of antisense oligonucleotide dosage and dosing frequency is within the abilities of those skilled in the art, and depends upon factors such as route of administration and animal body weight. Following a period of treatment with antisense oligonucleotides, RNA is isolated from liver tissue and changes in kallikrein nucleic acid expression are measured.

### Certain Indications

In certain embodiments, the invention provides methods of treating an individual comprising administering one or more pharmaceutical compositions of the present invention. In certain embodiments, the individual has a metabolic condition. In certain embodiments, the individual has a metabolic syndrome. In certain embodiments, the individual is at risk for developing a metabolic condition, including, but not limited to, metabolic syndrome, obesity, type I diabetes, or type II diabetes. In certain embodiments, the individual has been identified as in need of therapy. Examples of such individuals include, but are not limited to those having one or more symptoms or risk factors for having obesity, which include, inactivity, unhealthy diet and eating habits, lifestyle, quitting smoking, pregnancy, lack of sleep, certain medications, age, social and economic issues, and medical problems, such as, Prader-Willi syndrome, Cushing's syndrome, polycystic ovary syndrome, and arthritis. In certain embodiments, examples of such individuals include, but are not limited to those having one or more symptoms or risk factors for having type I diabetes, which include genetics and family history, diseases of the pancreas, and infection or illness. Examples of such individuals include, but are not limited to those having one or more symptoms or risk factors for having type II diabetes, which include, being overweight, impaired glucose tolerance or impaired fasting glucose, insulin resistance, ethnic background, hypertension, low levels of HDL “good” cholesterol and high triglyceride levels, history of gestational diabetes, inactivity, family history, polycystic ovary syndrome, and age over 45 years. In certain embodiments, provided herein are methods for prophylactically reducing kallikrein expression in an individual. Certain embodiments include treating an individual in need thereof by administering to an individual a therapeutically effective amount of an antisense compound targeted to a kallikrein nucleic acid.

In one embodiment, administration of a therapeutically effective amount of an antisense compound targeted to a kallikrein nucleic acid is accompanied by monitoring of kallikrein levels in the serum of an individual, to determine an individual's response to administration of the antisense compound. An individual's response to administration of the antisense compound is used by a physician to determine the amount and duration of therapeutic intervention.

In certain embodiments, administration of an antisense compound targeted to a kallikrein nucleic acid results in reduction of kallikrein expression by at least 15, 20, 25, 30, 35, 40, 45, 50, 55, 60, 65, 70, 75, 80, 85, 90, 95 or 99%, or a range defined by any two of these values. In certain embodiments, administration of an antisense compound targeted to a kallikrein nucleic acid results in a change in a measure of inflammation, swelling, hypertension, and/or vascular permeability. In certain embodiments, administration of a kallikrein antisense compound increases the measure by at least 15, 20, 25, 30, 35, 40, 45, 50, 55, 60, 65, 70, 75, 80, 85, 90, 95 or 99%, or a range defined by any two of these values. In some embodiments, administration of a kallikrein antisense compound decreases the measure by at least 15, 20, 25, 30, 35, 40, 45, 50, 55, 60, 65, 70, 75, 80, 85, 90, 95 or 99%, or a range defined by any two of these values.

In certain embodiments, pharmaceutical compositions comprising an antisense compound targeted to kallikrein are used for the preparation of a medicament for treating a patient suffering or susceptible to a metabolic condition including obesity, type I diabetes, and type II diabetes.

### Certain Combination Therapies

In certain embodiments, one or more pharmaceutical compositions described herein are co-administered with one or more other pharmaceutical agents. In certain embodiments, such one or more other pharmaceutical agents are designed to treat the same disease, disorder, or condition as the one or more pharmaceutical compositions described herein. In certain embodiments, such one or more other pharmaceutical agents are designed to treat a different disease, disorder, or condition as the one or more pharmaceutical compositions described herein. In certain embodiments, such one or more other pharmaceutical agents are designed to treat an undesired side effect of one or more pharmaceutical compositions described herein. In certain embodiments, one or more pharmaceutical compositions described herein are co-administered with another pharmaceutical agent to treat an undesired effect of that other pharmaceutical agent. In certain embodiments, one or more pharmaceutical compositions described herein are co-administered with another pharmaceutical agent to produce a combinational effect. In certain embodiments, one or more pharmaceutical compositions described herein are co-administered with another pharmaceutical agent to produce a synergistic effect.

In certain embodiments, one or more pharmaceutical compositions described herein and one or more other pharmaceutical agents are administered at the same time. In certain embodiments, one or more pharmaceutical compositions described herein and one or more other pharmaceutical agents are administered at different times. In certain embodiments, one or more pharmaceutical compositions described herein and one or more other pharmaceutical agents are prepared together in a single formulation. In certain embodiments, one or more pharmaceutical compositions described herein and one or more other pharmaceutical agents are prepared separately.

In certain embodiments, pharmaceutical agents that may be co-administered with a second agent described herein including metabolic agents. In certain embodiments, second agents include, but are not limited to, a glucose-lowering agent. The glucose lowering agent can include, but is not limited to, a therapeutic lifestyle change, PPAR agonist, a dipeptidyl peptidase (IV) inhibitor, a GLP-1 analog, insulin or an insulin analog, an insulin secretagogue, a SGLT2 inhibitor, a human amylin analog, a biguanide, an alpha-glucosidase inhibitor, or a combination thereof. The glucose-lowering agent can include, but is not limited to metformin, sulfonylurea, rosiglitazone, meglitinide, thiazolidinedione, alpha-glucosidase inhibitor or a combination thereof. The sulfonylurea can be acetohexamide, chlorpropamide, tolbutamide, tolazamide, glimepiride, a glipizide, a glyburide, or a gliclazide. The meglitinide can be nateglinide or repaglinide. The thiazolidinedione can be pioglitazone or rosiglitazone. The alpha-glucosidase can be acarbose or miglitol.

In some embodiments, the glucose-lowering therapeutic is a GLP-1 analog. In some embodiments, the GLP-1 analog is exendin-4 or liraglutide.

In other embodiments, the glucose-lowering therapeutic is a sulfonylurea. In some embodiments, the sulfonylurea is acetohexamide, chlorpropamide, tolbutamide, tolazamide, glimepiride, a glipizide, a glyburide, or a gliclazide.

In some embodiments, the glucose-lowering drug is a biguanide. In some embodiments, the biguanide is metformin, and in some embodiments, blood glucose levels are decreased without increased lactic acidosis as compared to the lactic acidosis observed after treatment with metformin alone.

In some embodiments, the glucose-lowering drug is a meglitinide. In some embodiments, the meglitinide is nateglinide or repaglinide.

In some embodiments, the glucose-lowering drug is a thiazolidinedione. In some embodiments, the thiazolidinedione is pioglitazone, rosiglitazone, or troglitazone. In some embodiments, blood glucose levels are decreased without greater weight gain than observed with rosiglitazone treatment alone.

In some embodiments, the glucose-lowering drug is an alpha-glucosidase inhibitor. In some embodiments, the alpha-glucosidase inhibitor is acarbose or miglitol.

In a certain embodiment, glucose-lowering therapy is therapeutic lifestyle change.

In certain embodiments, second agents include, but are not limited to, lipid-lowering agents. The lipid-lowering agent can include, but is not limited to atorvastatin, simvastatin, rosuvastatin, and ezetimibe. In certain such embodiments, the lipid-lowering agent is administered prior to administration of a pharmaceutical composition described herein. In certain such embodiments, the lipid-lowering agent is administered following administration of a pharmaceutical composition described herein. In certain such embodiments the lipid-lowering agent is administered at the same time as a pharmaceutical composition described herein. In certain such embodiments the dose of a co-administered lipid-lowering agent is the same as the dose that would be administered if the lipid-lowering agent was administered alone. In certain such embodiments the dose of a co-administered lipid-lowering agent is lower than the dose that would be administered if the lipid-lowering agent was administered alone. In certain such embodiments the dose of a co-administered lipid-lowering agent is greater than the dose that would be administered if the lipid-lowering agent was administered alone.

In certain embodiments, a co-administered lipid-lowering agent is a HMG-CoA reductase inhibitor. In certain such embodiments the HMG-CoA reductase inhibitor is a statin. In certain such embodiments the statin is selected from atorvastatin, simvastatin, pravastatin, fluvastatin, and rosuvastatin.

In certain embodiments, a co-administered lipid-lowering agent is a cholesterol absorption inhibitor. In certain such embodiments, cholesterol absorption inhibitor is ezetimibe.

In certain embodiments, a co-administered lipid-lowering agent is a co-formulated HMG-CoA reductase inhibitor and cholesterol absorption inhibitor. In certain such embodiments the co-formulated lipid-lowering agent is ezetimibe/simvastatin.

In certain embodiments, a co-administered lipid-lowering agent is a microsomal triglyceride transfer protein inhibitor (MTP inhibitor).

In certain embodiments, a co-administered lipid-lowering agent is an oligonucleotide targeted to ApoB.

In certain embodiments, second agents include, but are not limited to an anti-obesity drug or agent. Such anti-obesity agents include but are not limited to Orlistat, Sibutramine, or Rimonabant, and may be administered as described above as adipose or body weight lowering agents. In certain embodiments, the antisense compound may be co-administered with appetite suppressants. Such appetite suppressants include but are not limited to diethylpropion tenuate, mazindol, orlistat, phendimetrazine, phentermine, and sibutramine and may be administered as described herein. In certain embodiment, the anti-obesity agents are CNS based such as, but not limited to, sibutramine or GLP-1 based such as, but not limited to, liraglutide.

In certain embodiments, second agents include, but are not limited to an antipsychotic drug or agent. Such antipsychotic agents therapeutics may be administered as described above to reduce metabolic abnormalities associated with treatment with antipsychotic agents. In a particular embodiment administering of the Kallikrein inhibitor results indecreased body weight without affecting the CNS effects of the psychotherapeutic agent. Such antipsychotic agents include, but are not limited to clozapine, olanzapine, aripiprazole, risperidone and ziprasidone.

In certain embodiments, the pharmaceutical compositions of the present invention may be administered in conjunction with a lipid-lowering therapy. In certain such embodiments, a lipid-lowering therapy is therapeutic lifestyle change. In certain such embodiments, a lipid-lowering therapy is LDL apheresis.

In certain embodiments, pharmaceutical agents that may be co-administered with a kallikrein specific inhibitor described herein include, but are not limited to, an additional kallikrein inhibitor. In certain embodiments, the co-administered pharmaceutical agent is administered prior to administration of a pharmaceutical composition described herein. In certain embodiments, the co-administered pharmaceutical agent is administered following administration of a pharmaceutical composition described herein. In certain embodiments the co-administered pharmaceutical agent is administered at the same time as a pharmaceutical composition described herein. In certain embodiments the dose of a co-administered pharmaceutical agent is the same as the dose that would be administered if the co-administered pharmaceutical agent was administered alone. In certain embodiments the dose of a co-administered pharmaceutical agent is lower than the dose that would be administered if the co-administered pharmaceutical agent was administered alone. In certain embodiments the dose of a co-administered pharmaecutical agent is greater than the dose that would be administered if the co-administered pharmaceutical agent was administered alone.

In certain embodiments, the co-administration of a second compound enhances the metabolic effect of a first compound, such that co-administration of the compounds results in a metabolic effect that is greater than the effect of administering the first compound alone. In other embodiments, the co-administration results in metabolic effects that are additive of the effects of the compounds when administered alone. In certain embodiments, the co-administration results in metabolic effects that are supra-additive of the effects of the compounds when administered alone. In certain embodiments, the first compound is an antisense compound. In certain embodiments, the second compound is an antisense compound.

### EXAMPLES

### Non-Limiting Disclosure and Incorporation by Reference

While certain compounds, compositions, and methods described herein have been described with specificity in accordance with certain embodiments, the following examples serve only to illustrate the compounds described herein and are not intended to limit the same. Each of the references recited in the present application is incorporated herein by reference in its entirety.

### Example 1

### Antisense Inhibition of Murine Kallikrein B, Plasma (Fletcher Factor) 1 (KLKB1) mRNA in Mouse Primary Hepatocytes

Antisense oligonucleotides targeted to a murine KLKB1 nucleic acid were tested for their effect on KLKB1 mRNA in vitro. Cultured mouse primary hepatocytes were transfected using Cytofectin reagent with 12.5 nM, 25.0 nM, 50.0 nM, 100.0 nM, or 200.0 nM antisense oligonucleotide. After a treatment period of approximately 24 hours, RNA was isolated from the cells and mouse KLKB1 mRNA levels were measured by quantitative real-time PCR. Murine KLKB1 primer probe set RTS3313 (forward sequence TGCCTGCTGTTCAGCTTTCTC, designated herein as SEQ ID NO: 20; reverse sequence TGGCAAAGTCCCTGTAATGCT, designated herein as SEQ ID NO: 21; probe sequence CGTGACTCCACCCAAAGAGACAAATAAACG, designated herein as SEQ ID NO: 22) was used to measure mRNA levels. KLKB1 mRNA levels were adjusted according to total RNA content, as measured by RIBOGREEN.

Two of the antisense oligonucleotides tested in the assay, ISIS 482585 (TGTGTCAGCTTTGGAAGGCA; SEQ ID NO: 23) and ISIS 482584 (GGCATATTGGTTTTTGGAAT; SEQ ID NO: 24), were designed as 5-10-5 MOE gapmers, and are 20 nucleosides in length, wherein the central gap segment is comprised of ten 2′-deoxynucleosides and is flanked on both sides (in the 5′ and 3′ directions) by wings comprising 5 nucleosides each. Each nucleoside in the 5′ wing segment and each nucleoside in the 3′ wing segment has a 2′-MOE modification. The internucleoside linkages throughout the gapmer are phosphorothioate (P═S) linkages. All cytosine residues throughout the gapmer are 5-methylcytosines. ISIS 482585 is targeted to nucleobases 1606 to 1625 of mouse KLKB1 mRNA (GENBANK Accession No. NM_{—}008455.2, incorporated herein as SEQ ID NO: 1). ISIS 482584 is targeted to nucleobases 1586 to 1605 of SEQ ID NO: 1.

KLKB1 mRNA levels were significantly reduced in a dose-dependent manner in ISIS oligonucleotide-treated cells. The data is presented in Table 1, expressed as percent inhibition compared to control untreated cells.

TABLE 1
Dose-dependent inhibition of murine KLKB1 mRNA
in mouse primary hepatocytes

| | | | | | 200.0 | IC₅₀ |
|---|---|---|---|---|---|---|
| ISIS No | 12.5 nM | 25.0 nM | 50.0 nM | 109.0 nM | nM | (nM) |
| 482584 | 0 | 36 | 17 | 60 | 83 | 87 |
| 482585 | 0 | 0 | 33 | 65 | 86 | 79 |

### Example 2

### Effect of Antisense Inhibition of Murine KLKB1 in Diet-Induced Obese Mice

The DIO mouse model is a standard model for studying obesity and other metabolic-related diseases (Surwit, R. et al., Mouse Genome. 92: 523-525, 1994). Metabolic endpoints of treatment with ISIS 482585 were evaluated in DIO mice.

### Treatment

C57BL/6 mice were maintained on a 12-hour light/dark cycle and fed ad libitum a high fat diet for a period of 4 weeks. Antisense oligonucleotides were prepared in PBS and sterilized by filtering through a 0.2 micron filter. Oligonucleotides were dissolved in 0.9% PBS for infection.

At the end of 4 weeks, the mice were divided into three treatment groups, based on body weight and body fat content. The first group was injected subcutaneously with ISIS 482585 at a dose of 50 mg/kg/week for 9 weeks. The second group was injected subcutaneously with control oligonucleotide ISIS 141923 (CCTTCCCTGAAGGTTCCTCC, 5-10-5 MOE gapmer with no known murine target sequence (SEQ ID NO: 25)) at a dose of 50 mg/kg/week for 9 weeks. The third group was injected subcutaneously with PBS for 9 weeks. The PBS group served as the control to which the first two groups were compared. The high-fat diet was administered for the entire study period.

### Inhibition of KLKB1 mRNA

Twenty four hours after the final dose, the animals were sacrificed and livers were harvested. RNA was isolated for real-time RT-PCR analysis of KLKB1. Treatment with ISIS 482585 reduced murine KLKB1 mRNA by 91% compared to the control group. Treatment with the control oligonucleotide did not reduce murine KLKB1 mRNA by any significant amount, as expected.

### Effect on Food Intake

The cumulative food intake per cage of each group was monitored weekly and is presented in Table 2. As indicated in Table 2, the food intake of the mice treated with ISIS 482585 is slightly reduced compared to the PBS control group.

TABLE 2
Cumulative food intake (g/cage)

| | Week 1 | Week 2 | Week 3 | Week 4 | Week 5 | Week 6 | Week 7 | Week 8 | Week 8.5 |
|---|---|---|---|---|---|---|---|---|---|
| PBS | 55 | 106 | 158 | 220 | 264 | 318 | 369 | 430 | 462 |
| ISIS 141923 | 57 | 109 | 161 | 221 | 265 | 318 | 369 | 428 | 456 |
| ISIS 482585 | 55 | 104 | 153 | 208 | 246 | 293 | 339 | 391 | 417 |

### Effect on Body-Weight

The body weight of individual mice of each group was monitored weekly and the average weight per group is presented in Table 3. As indicated in Table 3, there was no change in the body weight of the mice treated with ISIS 482585 compared to the baseline values, whereas the weight in both the PBS control and the ISIS 141923 treated groups increased throughout the study period compared to the baseline.

TABLE 3
Body weight (g)

| | Week 1 | Week 2 | Week 3 | Week 4 | Week 5 | Week 6 | Week 7 | Week 8 | Week 9 |
|---|---|---|---|---|---|---|---|---|---|
| PBS | 33 | 34 | 35 | 36 | 38 | 39 | 40 | 41 | 42 |
| ISIS 141923 | 34 | 35 | 35 | 36 | 38 | 39 | 38 | 40 | 40 |
| ISIS 482585 | 33 | 34 | 34 | 34 | 34 | 34 | 34 | 34 | 34 |

### Effect on Body Fat Content and Fat Depot Weight

Whole body fat content was measured using an Echo MRI system (Echo Medical System, Houston, Tex.). The data is presented in Table 4, expressed in grams, as well as in Table 5, expressed as a percentage of the body weight. The data indicates that mice treated with ISIS 482585 had significantly less body fat content compared to the control group.

Both epididymal and perirenal white adipose depots from all mice groups were dissected and weighed. The data is presented in Table 6, expressed in grams. The data indicates that mice treated with ISIS 482585 had significantly less fat depot weight compared to the control groups.

TABLE 4
Whole fat content (g)

| | Week 0 | Week 4 | Week 7 | Week 9 |
|---|---|---|---|---|
| PBS | 4.9 | 8.8 | 12.3 | 14.1 |
| ISIS 141923 | 5.0 | 9.4 | 10.7 | 10.6 |
| ISIS 482585 | 5.0 | 6.9 | 6.5 | 5.9 |

TABLE 5
Whole fat content (% body weight)

| | Week 0 | Week 4 | Week 7 | Week 9 |
|---|---|---|---|---|
| PBS | 16 | 25 | 30 | 32 |
| ISIS 141923 | 16 | 25 | 27 | 25 |
| ISIS 482585 | 16 | 20 | 19 | 17 |

TABLE 6
Fat depot weight (g)

| | Epididymal | Perirenal |
|---|---|---|
| PBS | 2.4 | 1.0 |
| ISIS 141923 | 1.8 | 0.7 |
| ISIS 482585 | 0.9 | 0.3 |

### Effect Glucose and Insulin Levels

Plasma glucose values were determined using a clinical analyzer (Olympus AU400, Olympus American Inc., Melville, N.Y.). Plasma insulin concentrations were determined by a RIA Assay system (Linco). The results are presented in Tables 7 and 8. The data demonstrates that both glucose and insulin were significantly reduced on treatment with ISIS 482585 compared to the control groups.

TABLE 7
Plasma glucose (mg/dL)

| | Week 0 | Week 8 |
|---|---|---|
| PBS | 216 | 242 |
| ISIS 141923 | 187 | 217 |
| ISIS 482585 | 217 | 184 |

TABLE 8
Plasma insulin (ng/mL)

| | Week 8 |
|---|---|
| PBS | 3.7 |
| ISIS 141923 | 2.7 |
| ISIS 482585 | 1.2 |

### Effect Lipid Levels

Plasma concentrations of cholesterol and triglycerides were measured using a clinical analyzer (Olympus AU400, Olympus American Inc, Melville, N.Y.). The results are presented in Tables 9 and 10, expressed in mg/dL. The data indicate that treatment with ISIS 482585 decreased plasma triglycerides compared to the control groups.

TABLE 8
Plasma cholesterol (mg/dL)

| | Week 0 | Week 3 | Week 8 |
|---|---|---|---|
| PBS | 163 | 190 | 206 |
| ISIS 141923 | 192 | 192 | 192 |
| ISIS 482585 | 159 | 222 | 210 |

TABLE 9
Plasma triglycerides (mg/dL)

| | Week 0 | Week 3 | Week 8 |
|---|---|---|---|
| PBS | 105 | 113 | 105 |
| ISIS 141923 | 92 | 89 | 90 |
| ISIS 482585 | 92 | 89 | 66 |

### Evaluation of Liver Function

To evaluate the impact of ISIS oligonucleotides on the hepatic function of the mice, plasma concentrations of transaminases were measured using a clinical analyzer (Olympus AU400, Olympus American Inc, Melville, N.Y.). Measurements of alanine transaminase (ALT) and aspartate transaminase (AST) are expressed in IU/L. The results are presented is Tables 10 and 11.

TABLE 10
Plasma ALT (IU/L)

| | Week 0 | Week 3 | Week 8 |
|---|---|---|---|
| PBS | 32 | 39 | 51 |
| ISIS 141923 | 35 | 34 | 45 |
| ISIS 482585 | 30 | 29 | 266 |

TABLE 11
Plasma AST (IU/L)

| | Week 0 | Week 3 | Week 8 |
|---|---|---|---|
| PBS | 50 | 50 | 61 |
| ISIS 141923 | 51 | 48 | 54 |
| ISIS 482585 | 49 | 50 | 205 |

### Effect on Organ Weight

Weights of the livers, spleens and kidneys of the mice were taken at the end of the study period and are presented in Table 12. The data indicates that there was no significant difference in organ weights. Hence, treatment with the ISIS 482385 was tolerable to the mice in terms of their organ weights.

TABLE 12
Organ weights (g)

| | Kidney | Spleen | Liver |
|---|---|---|---|
| PBS | 0.37 | 0.10 | 1.63 |
| ISIS 141923 | 0.37 | 0.13 | 1.56 |
| ISIS 482585 | 0.37 | 0.16 | 1.56 |

### Example 3

### Effect of Antisense Inhibition of Murine KLKB1 in Mice Kept on a High-Fat Diet

The DIO mouse model is a standard model for studying obesity and other metabolic-related diseases (Surwit, R. et al., Mouse Genome. 92: 523-525, 1994). Metabolic endpoints after treatment with ISIS 482584, targeting KLKB1 mRNA, were evaluated in DIO mice, as well as in mice kept on normal chow diet.

### Treatment

Male C57BL/6 mice, 8 weeks of age were obtained from Jackson Laboratories (Bar Harbor, Me.). Mice received either normal chow or a high fat diet containing 60% fat (Research Diets). Antisense oligonucleotides were prepared in PBS and sterilized by filtering through a 0.2 micron filter. Oligonucleotides were dissolved in sterile PBS for injection.

The DIO mice were divided into two treatment groups of 4 mice each. The normal chow mice were also divided into two treatment groups of 4 mice each. One DIO mouse group and one normal chow group were injected subcutaneously with ISIS 482584 at a dose of 40 mg/kg every 4 days for 4 weeks. The second group of DIO mice and normal chow mice were injected subcutaneously with control oligonucleotide ISIS 141923 at a dose of 40 mg/kg/week every 4 days for 20 weeks.

### Effect on Body Weight

The body weight of individual mice in each group was monitored weekly and the average weight per group is presented in Table 13. As indicated in Table 13, there was no change in the body weight of the DIO mice treated with ISIS 482584 compared to the baseline. The weight in the control DIO group increased throughout the study period compared to the baseline. The increase in body weight of normal chow-fed mice treated with ISIS 482584 was also less than that of the normal chow-fed control group.

Hence, antisense inhibition of KLKB1 mRNA reduced body weight in DIO mice and normal chow-fed mice compared to the control groups.

TABLE 13
Body weight (g) to DIO mice and normal chow-fed mice

| Mouse | | | | | | |
|---|---|---|---|---|---|---|
| strain | Treatment | Day 4 | Day 8 | Day 14 | Day 22 | Day 26 |
| DIO mice | ISIS 482584 | 22.1 | 23.3 | 22.7 | 23.0 | 23.8 |
| | ISIS 141923 | 22.5 | 25.1 | 25.9 | 28.0 | 29.2 |
| Normal | ISIS 482584 | 23.7 | 24.4 | 25.0 | 25.1 | 25.5 |
| chow-fed | ISIS 141923 | 23.3 | 24.6 | 26.2 | 27.7 | 28.7 |
| mice | | | | | | |

### Example 4

### Effect of Antisense Inhibition of Murine KLKB1 in Ob/Ob Mice

Ob/ob mice are homozygous for the obese spontaneous mutation (Lep^{ob}, commonly referred to as ob or ob/ob) and exhibit obesity, hyperphagia, hyperglycemia, and elevated plasma insulin. The effect of ISIS 482584 targeting KLKB1 on various metabolic end-points was evaluated in ob/ob mice.

### Treatment

Male ob/ob mice, 6 weeks of age, were obtained from Jackson Laboratories (Bar Barber, Me.). Antisense oligonucleotides were prepared in PBS and sterilized by filtering through a 0.2 micron filter. Oligonucleotides were dissolved In sterile PBS for injection.

The mice were divided into two treatment groups of 8 mice each. Measurements taken at the start of the study period are noted as baseline measurements. The first group was injected subcutaneously with ISIS 482584 at a dose of 40 mg/kg every 4 days for 4 weeks. The second group was injected subcutaneously with control oligonucleotide ISIS 141923 at a dose of 40 mg/kg/week every 4 days for 4 weeks.

### Effect on Body Weight

Body weights were measured at baseline and at one week intervals throughout the study period. The data is presented in Table 14 and demonstrated that antisense inhibition of KLKB1 in this model significantly reduced body weight compared to the control group. The body weight following ISIS 482584 treatment increased from 37 g to 45 g (p<0.001), whereas the body weight following the control oligonucleotide treatment increased from 36 g to 50 g (p<0.001). Body weight was significantly different between groups on week 3 (p=0.049) and week 4 (p=0.024).

TABLE 14
Body weights (g) in ob/ob mice

| ISIS No | Baseline | Week 1 | Week 2 | Week 3 | Week 4 |
|---|---|---|---|---|---|
| 482584 | 37 | 40 | 43 | 44 | 45 |
| 141923 | 36 | 40 | 45 | 48 | 50 |

### Effect on Body Fat

Body composition was measured by Dual Energy X-ray Absorptiometry (DEXA) using a PIXImus II densitometer (GE Lunar, Madison, Wis.) at baseline (day 0) and after 4 weeks of treatment (day 28). The data is presented in Table 15, expressed as grams. Total body fat following antisense oligonucleotide treatment increased from 20 g to 25 g, while control oligonucleotide treatment increased body fat from 18 g to 31 g. Hence, antisense inhibition of KLKB1 mRNA reduced body fat composition in this model compared to the control group.

TABLE 15
Total body fat (g) of ob/ob mice

| ISIS No | Day 0 | Day 28 |
|---|---|---|
| 482584 | 20.7 | 25.2 |
| 141923 | 18.4 | 31.3 |

### Effect on Glucose Levels

Glucose levels in the blood were measured with a One Touch Ultra Glucometer using blood from the tail of mice in a fed condition. Measurements were taken at baseline and at one week intervals throughout the study period. The data is presented in Table 16, expressed in mg/dL. Blood glucose levels following antisense oligonucleotide treatment decreased significantly (p=0.023) compared to baseline values, while glucose levels in the control group increased throughout the treatment period.

Glucose levels were also measured in the fasting state by measuring the levels in the morning after an overnight fast. The data is presented in Table 17, expressed in mg/dL. Blood glucose levels on week 4 following antisense oligonucleotide treatment decreased significantly (p=0.026) compared to the control group.

Hence, antisense inhibition of KLKB1 mRNA reduced glucose levels in this model compared to the control group.

TABLE 16
Glucose levels in the fed state in ob/ob mice (mg/dL)

| ISIS No | Baseline | Week 1 | Week 2 | Week 3 | Week 4 |
|---|---|---|---|---|---|
| 482584 | 399 | 298 | 258 | 232 | 241 |
| 141923 | 403 | 388 | 469 | 399 | 436 |

TABLE 17
Glucose levels in the fasting state in ob/ob mice (mg/dL)

| ISIS No | Week 4 |
|---|---|
| 482584 | 141 |
| 141923 | 247 |

### Effect on Plasma Triglyceride Levels

Blood samples were collected at baseline and at one week intervals throughout the study period. Samples were collected from tail snips using heparinized capillary tubes. The collected blood was then centrifuged at 3,000 rpm for 5 min to collect plasma. Triglyceride levels were measured using a colorimetric assay kit (#10010303; Cayman Chemical Co., Ann Arbor, Mich.). The data is presented in Table 18, expressed in mg/dL. Plasma triglycerides following antisense oligonucleotide treatment decreased compared to the control group.

Hence, antisense inhibition of KLKB1 mRNA reduced plasma triglyceride levels in this model compared to the control group.

TABLE 18
Triglyceride levels in ob/ob mice (mg/dL)

| ISIS No | Baseline | Week 1 | Week 2 | Week 3 |
|---|---|---|---|---|
| 482584 | 346 | 88 | 69 | 84 |
| 141923 | 240 | 309 | 285 | 268 |

### Example 5

### Effect of Antisense Inhibition of Murine KLKB1 in Ob/Ob Mice and Wild-Type Mice

The effect of ISIS 482584 targeting KLKB1 on various metabolic end-points was evaluated in ob/ob mice, as well as wild-type mice.

### Treatment

Male ob/ob mice, 6 weeks of age, and age-matched wild-type mice were obtained from Jackson Laboratories (Bar Harbor, Me.). Antisense oligonucleotides were prepared in PBS and sterilized by filtering through a 0.2 micron filter. Oligonucleotides were dissolved in 0.9% PBS for injection.

The ob/ob mice were divided into two treatment groups of 4 mice each. The wild-type mice were divided into two treatment groups of 4 mice each. Measurements taken at the start of the study period are noted as baseline measurements. One group of ob/ob mice and one group of wild-type mice were injected subcutaneously with ISIS 482584 at a dose of 40 mg/kg every 4 days for 5 weeks. The second groups of ob/ob mice and wild-type mice were injected subcutaneously with control oligonucleotide ISIS 141923 at a dose of 40 mg/kg/week every 4 days for 5 weeks.

### Effect on Plasma Insulin Levels

Blood samples were collected after 3 weeks from mice in the fed condition via tail snips using heparinized capillary tubes. Blood samples were also collected after 5 weeks from mice in the fasting condition. The collected blood was then centrifuged at 3,000 rpm for 15 min at 4° C. to collect plasma. Insulin concentrations were measured using an insulin ELISA kit (#90080; Cayman Chemical Co., Ann. Arbor, Mich.). The data is presented in Table 19, expressed in ng/mL.

Plasma insulin levels following antisense oligonucleotide treatment in the fed ob/ob mice and wild-type mice significantly decreased compared to the respective control mice groups (p=0.05 for ob/ob mice and p=0.008 for wild-type mice). Plasma insulin levels between the mice strains in the fed state was also significantly different (p<0.01). In the fasting condition, plasma insulin levels were significantly decreased between the mouse strains treated with ISIS 482584 compared to the respective control groups (p=0.05).

Hence, antisense inhibition of KLKB1 mRNA reduced plasma insulin levels in this model compared to the control group.

TABLE 19
Plasma insulin levels in ob/ob mice and wild-type mice (ng/mL)

| | |
|---|---|
| | Fasting state (5 |
| Fed state (3 weeks) | weeks) |

| ISIS No | ob/ob | Wild-type | ob/ob | Wild-type |
|---|---|---|---|---|
| 482584 | 3.8 | 0.7 | 2.5 | 0.9 |
| 141923 | 8.5 | 1.4 | 2.6 | 0.8 |

### Effect on Glucose Levels Measured by an Intraperitoneal Glucose Tolerance Test

Glucose levels in the blood were measured from the tail of mice in a fasting condition with a One Touch Ultra Glucometer. After baseline measurements, 20% dextrose at 2 g/kg was injected by intraperitoneal injection and blood glucose was measured at 15 min, 30 min, 60 min and 120 min after the injection. The data is presented in Table 20, expressed in mg/dL. Blood glucose levels in ob/ob mice following antisense oligonucleotide treatment were significantly decreased compared to the ob/ob control group (p<0.05). Glucose levels of ob/ob mice treated with ISIS 482584 were comparable to that of wild-type mice, as presented in Table 20. ‘n.d.’ indicates that there is no data for that particular time point.

Hence, antisense inhibition of KLKB1 mRNA led to greater glucose tolerance in ob/ob mice compared to the control group.

TABLE 20
Glucose levels in ob/ob mice and wild-type mice (mg/dL)

| Mouse | | | | | | |
|---|---|---|---|---|---|---|
| strain | Treatment | Baseline | 15 min | 30 min | 60 min | 120 min |
| Ob/ob | ISIS 482584 | 112 | 374 | 350 | 245 | 155 |
| mice | ISIS 141923 | 205 | 581 | 600 | 600 | 529 |
| Wild- | ISIS 482584 | 120 | 224 | 202 | 169 | n.d |
| type | ISIS 141923 | 125 | 327 | 327 | 256 | n.d |
| mice | | | | | | |

### Example 5

### Effect of Antisense Inhibition of Murine KLKB1 on Glucose Tolerance in Ob/Ob Mice

The effect of ISIS 482584 targeting KLKB1 on glucose tolerance was evaluated in ob/ob mice.

### Treatment

Male ob/ob mice, 6 weeks of age were obtained from Jackson Laboratories (Bar Harbor, Me.). Antisense oligonucleotides were prepared in PBS and sterilized by filtering through a 0.2 micron filter. Oligonucleotides were dissolved in sterile PBS for injection.

The ob/ob mice were divided into two treatment groups of 3-4 mice each. Measurements taken at the start of the study period are noted as baseline measurements. The first group was injected subcutaneously with ISIS 482584 at a dose of 40 mg/kg every 4 days for 5 weeks. The second group of mice was injected subcutaneously with control oligonucleotide ISIS 141923 at a dose of 40 mg/kg/week every 4 days for 5 weeks.

### Effect on Glucose Levels Measured by an Oral Glucose Tolerance Test

After 5 weeks of treatment, the mice were fasted overnight. Blood glucose was measured in the morning via tail snip using a Once Touch Ultra Glucometer. After baseline measurements, 20% dextrose at 2 g/kg was given via oral gavage using a Teflon 18-gauge feeding needle. Blood glucose was then monitored at 15 min, 30 min, 60 min, and 120 min after injection. The data is presented in Table 21, expressed as mg/dL. At 120 min, blood glucose levels in ob/ob mice following antisense oligonucleotide treatment were reduced to near baseline values, whereas the blood glucose levels of the control group were still significantly elevated compared to the baseline level.

Hence, antisense inhibition of KLKB1 mRNA led to greater glucose tolerance in ob/ob mice compared to the control group.

TABLE 21
Glucose levels in ob/ob mice (mg/dL)

| ISIS No | Baseline | 15 min | 30 min | 60 min | 120 min |
|---|---|---|---|---|---|
| 482584 | 171 | 495 | 584 | 457 | 252 |
| 141923 | 288 | 598 | 600 | 600 | 471 |

### Effect on Insulin Levels Measured by an Intraperitoneal Insulin Tolerance Test

After 4 weeks of treatment, the mice were allowed overnight food and water. Blood glucose was measured in the afternoon via tail snip using a Once Touch Ultra Glucometer. After baseline measurements, 0.1 mU/kg of regular insulin was given via intraperitoneal injection. Blood glucose was then monitored at 15 min, 30 min, 60 min, and 120 min after injection. The data is presented in Table 22, expressed as mg/dL. Blood glucose levels in ob/ob mice following antisense oligonucleotide treatment were decreased compared to the control group.

Hence, antisense inhibition of KLKB1 mRNA led to greater insulin tolerance in ungenotyped wild type mice compared to the control treatment group.

TABLE 22
Glucose levels in ungenotyped WT mice (mg/dL)

| ISIS No | Baseline | 15 min | 30 min | 45 min | 60 min |
|---|---|---|---|---|---|
| 482584 | 131 | 91 | 62 | 42 | 32 |
| 141923 | 181 | 109 | 107 | 85 | 74 |

### Example 6

### Effect of Antisense Inhibition of Murine KLKB1 in STZ-induced Diabetic Mice and Non-Diabetic Mice

Streptozotocin (STZ) is a naturally occurring chemical that is toxic to the insulin-producing beta cells of the pancreas. It is extensively used to induce diabetes in rodents (Wang, Z., et al., Diabetes. 47: 50-56, 1998). The effect of ISIS 482584 targeting KLKB1 on plasma glucose levels was evaluated in STZ-induced diabetic mice as well as in non-diabetic controls.

### Treatment

Male C57BL/6 mice, 8 weeks of age were obtained from Taconic Farms (Germantown, N.Y.). Diabetes was induced after a two-hour fast by intraperitoneal injection for 5 consecutive days of streptozotocin at 45 mg/kg in 50 mM sodium citrate (pH 4.5). On day 8, diabetes was confirmed by testing blood glucose levels using a One Touch Ultra Glucometer. Antisense oligonucleotides were prepared in PBS and sterilized by filtering through a 0.2 micron filter. Oligonucleotides were dissolved in 0.9% PBS for injection.

The STZ-induced diabetic mice were divided into three treatment groups of 4 mice each. Measurements taken at the start of the study period are noted as baseline measurements. The first group was injected subcutaneously with ISIS 482584 at a dose of 40 mg/kg every 4 days for 20 weeks. The second group of mice was injected subcutaneously with control oligonucleotide ISIS 141923 at a dose of 40 mg/kg/week every 4 days for 20 weeks. The third group was injected subcutaneously with PBS every 4 days for 20 weeks. Two groups of C57BL/6 mice not treated with STZ were also included in this study as controls. The first group of control mice was injected subcutaneously with ISIS 482584 at a dose of 40 mg/kg every 4 days for 20 weeks. The second group of mice was injected subcutaneously with control oligonucleotide ISIS 141923 at a dose of 40 mg/kg/week every 4 days for 20 weeks.

### Effect on Glucose Levels

Blood glucose was measured at baseline and then at one week intervals via tail snip using a Once Touch Ultra Glucometer. The data is presented in Table 23, expressed as mg/dL. Blood glucose levels in the mice following antisense oligonucleotide treatment were significantly decreased compared to the control oligonucleotide group at week 3 (p=0.07), week 7 (p=0.025) and week 11 onwards (p=0.004).

Hence, antisense inhibition of KLKB1 mRNA led to reduction in glucose levels in STZ-induced diabetic mice compared to the control group.

TABLE 23
Glucose levels in STZ-induced diabetic mice (mg/dL)

| | | | | | | Week | Week |
|---|---|---|---|---|---|---|---|
| ISIS No | Baseline | Week 3 | Week 5 | Week 7 | Week 8 | 11 | 15 |
| 482584 | 340 | 403 | 352 | 408 | 277 | 177 | 202 |
| 141923 | 389 | 541 | 531 | 573 | 553 | 563 | 533 |
| PBS | 394 | 505 | 459 | 435 | 536 | 524 | 551 |

### Effect on Plasma Triglyceride Levels

Blood samples were collected at week 22. Samples were collected from tail snip using heparinized capillary tubes. The collected blood was then centrifuged at 3,000 rpm for 5 min to collect plasma. Triglyceride levels were measured using a colorimetric assay kit (#10010303; Cayman Chemical Co., Ann Arbor, Mich.). The data is presented in Table 24, expressed in mg/dL. Plasma triglycerides following antisense oligonucleotide treatment in the STZ-induced diabetic mice decreased compared to the control oligonucleotide treated STZ group, as well as the untreated STZ group (p<0.01). In case of the wild-type mice, the group treated with ISIS 482584 also had reduced triglyceride levels compared to the corresponding control group (p<0.01).

Hence, antisense inhibition of KLKB1 mRNA reduced plasma triglyceride levels compared to the control groups.

TABLE 24
Triglyceride levels in STZ-induced diabetic
mice and non-diabetic mice (mg/dL)

| Mouse strain | Treatment | Triglyceride levels |
|---|---|---|
| Diabetic mice | ISIS 482584 | 26 |
| | ISIS 141923 | 115 |
| | PBS | 163 |
| Wild-type | ISIS 482584 | 76 |
| mice | ISIS 141923 | 23 |

### Effect on Plasma Insulin Levels

Blood samples were collected at week 20 after overnight fasting from tail snip using heparinized capillary tubes. The collected blood was then centrifuged at 3,000 rpm for 15 min at 4° C. to collect plasma. Insulin concentrations were measured using an insulin ELISA kit (#90080; Cayman Chemical Co., Ann Arbor, Mich.). The data is presented in Table 25, expressed in ng/mL. Plasma insulin levels were not affected by antisense oligonucleotide treatment in the STZ-induced diabetic mice.

TABLE 25
Insulin levels in STZ-induced diabetic
mice and non-diabetic mice (ng/mL)

| Mouse strain | Treatment | Insulin levels |
|---|---|---|
| Diabetic mice | ISIS 482584 | 0.11 |
| | ISIS 141923 | 0.08 |
| | PBS | 0.08 |
| Wild-type | ISIS 482584 | 2.88 |
| mice | ISIS 141923 | 0.20 |

### Example 7

### Effect of Antisense Inhibition of Murine KLKB1 in Diet-Induced Obese Mice

Metabolic endpoints of treatment with ISIS 432584 were evaluated in DIO mice.

### Treatment

Male C57BL/6 mice at 8 weeks of age were maintained on a 12-hour light/dark cycle and fed ad libitum either a high fat diet providing 60 kcal % fat (D12492 Research Diets) or a normal chow diet.

The mice from each diet set were divided into two treatment groups, based on body weight and body fat content. One group from the high fat diet set and one group from the normal chow diet set were injected subcutaneously with ISIS 482584 at a dose of 40 mg/kg twice a week for 16 weeks. The second group from the high-fat diet set and from the normal chow diet set was injected subcutaneously with control oligonucleotide ISIS 141923 at a dose of 40 mg/kg twice a week for 16 weeks. The high-fat diet or normal chow diet was administered for the entire study period to the relevant mice. KLKB1 protein depletion was assessed by western blot analysis of the plasma of the ISIS oligonucleotide-treated mice and calculated to be >90% depleted compared to the control groups.

### Effect on Body Weight

The body weights of the mice in each group were measured at baseline (day 0) and at week 8 and are presented in Table 26. As indicated in Table 26, the average body weight of the mice treated with ISIS 482584 was reduced compared to the control groups.

TABLE 26
Body weights (g)

| | Normal | Normal | High-fat | High-fat |
|---|---|---|---|---|
| | diet, ISIS | diet, ISIS | diet, ISIS | diet, ISIS |
| | 141923 | 482584 | 141923 | 482584 |
| | treated | treated | treated | treated |
| Week 0 | 22.9 | 23.0 | 22.7 | 22.8 |
| Week 8 | 31.6 | 25.6 | 34.9 | 23.1 |

### Effect on Body Fat

Total body fat composition was measured by Dual Energy X-ray Absorptiometry (DEXA) using a PIXImus II densitometer (GE Lunar, Madison, Wis.) at baseline (day 0) and after 8 weeks of treatment. The data is presented in Table 27, expressed as grams. As indicated in Table 27, antisense inhibition of KLKB1 mRNA reduced body fat composition in the mice compared to the control group.

TABLE 27
Total body fat (g)

| | Normal | | | |
|---|---|---|---|---|
| | diet, ISIS | Normal | High-fat | High-fat |
| | 141923 | diet, ISIS | diet, ISIS | diet, ISIS |
| | treated | 482584 treated | 141923 treated | 482584 treated |
| Week 0 | 2.8 | 2.8 | 2.5 | 2.9 |
| Week 8 | 8.0 | 3.2 | 13.2 | 3.4 |

### Effect on Physical Activity

Activity was measured by using CLAMS (Comprehensive Lab Animal Monitoring System) after 8 weeks of treatment. The data is presented in Table 29, expressed as counts. The CLAMS detects motion in the x and y directions and increments a movement from 1 sensor to the next as a “count”. Motion is monitored for 50 seconds per chamber and re-measured every 10 minutes over a 24 hour cycle. As indicated is Table 28, antisense inhibition of KLKB1 mRNA did not have any effect on the mice compared to the control group.

TABLE 28
Average 12-hour activity (counts)

| | Normal Diet | High-fat Diet | High-fat Diet |
|---|---|---|---|
| | ISIS 141923 | ISIS 141923 | ISIS 482584 |
| | Treated | Treated | Treated |
| Light Cycle | 32 | 23 | 23 |
| Dark Cycle | 86 | 89 | 64 |

### Effect on Plasma Triglyceride Levels

Blood samples of mice in the fed and fasted condition were collected from tail snip using heparinized capillary tubes. The collected blood was then centrifuged at 3,000 rpm for 5 min to collect plasma. Triglyceride levels were measured using a colorimetric assay kit (#10010303; Cayman Chemical Co., Ann Arbor, Mich.). The data of the fed mice at baseline (day 0) and after 3 weeks is presented in Table 29, expressed in mg/dL. The data of the mice fasted for 12 hours at 11 weeks of treatment is presented in Table 30, expressed in mg/dL. Plasma triglycerides following antisense oligonucleotide treatment in the mice decreased compared to the control oligonucleotide treated group after 3 weeks in fed state. In case of the fasted mice, the high-fat diet group treated with ISIS 482584 also had reduced triglyceride levels compared to the corresponding control group.

Hence, antisense inhibition of KLKB1 mRNA reduced plasma triglyceride levels compared to the control groups.

TABLE 29
Triglyceride levels in the fed state (mg/dL)

| | Normal | | | |
|---|---|---|---|---|
| | diet, ISIS | Normal | High-fat | High-fat |
| | 141923 | diet, ISIS | diet, ISIS | diet, ISIS |
| | treated | 482584 treated | 141923 treated | 482584 treated |
| Week 0 | 166 | 172 | 159 | 169 |
| Week 3 | 93 | 56 | 74 | 47 |

TABLE 30
Triglyceride levels in the fasted state (mg/dL)

| | Normal | | | |
|---|---|---|---|---|
| | diet, ISIS | Normal | High-fat | High-fat |
| | 141923 | diet, ISIS | diet, ISIS | diet, ISIS |
| | treated | 482584 treated | 141923 treated | 482584 treated |
| Week 11 | 70 | 56 | 83 | 41 |

### Example 8

### Effect of Antisense Inhibition of Murine KLKB1 on Retinal and Systemic Parameters in a Hypertensive Rodent Model

The effect of treatment with ISIS 482584 on mice subjected to Angiotensin II (Ang II)-induced hypertension (Phipps, J. A. et al., Hypertension. 2009. 53: 175-181; Gao, B. B., et al., Nat. Med. 2007, 13: 181-188).

### Treatment

Male C57Bl/6 mice at 8 weeks of age were pre-treated with subcutaneous injections at 40 mg/kg of ISIS 482584 or control oligonucleotide ISIS 141923, administered twice a week for 3 weeks. At the end of 3 weeks, the mice had subcutaneous implantation of an osmotic pump, containing angiotensin II or phosphate buffered saline. Osmotic pumps (Alzet 1007D, 0.5 μL/hr) containing Angiotensin II at 2.88 μg/μL delivered Ang-II at 1153 μg/kg/d. Systemic blood pressure was measured by tail-cuff (Visitech 2000) at 3 days after pump implantation. Increased blood pressure confirmed Ang-II induction of hypertension. Retinal vascular permeability was measured at 5 days after pump implantation by Evans-blue albumin permeation.

### Effect on Body Weight

The body weights of all the mice were measured at weekly. The results are presented in Table 31, expressed in grams. The data indicates that treatment with ISIS 482584 prevented body weight gain in the mice.

TABLE 31
Body weights (g)

| | Week 0 | Week 1 | Week 2 | Week 3 |
|---|---|---|---|---|
| ISIS 141923 | 26.2 | 27.3 | 28.5 | 29.2 |
| ISIS 482584 | 25.5 | 25.9 | 25.8 | 25.8 |

### Effect on Blood Pressure

Systolic and diastolic blood pressure and heart rate of all the mice were measured at day 3 after implantation of osmotic pumps. The results are presented in Table 32, expressed in mm Hg and BPM.

TABLE 32
Systolic and Diastolic Blood Pressure
(mm Hg) and Heart Rate (beats/min)

| ISIS No | Pump | SBP | DBP | HR |
|---|---|---|---|---|
| 141923 | PBS | 102 | 84 | 601 |
| 141923 | ANG-II | 129 | 117 | 568 |
| 482584 | PBS | 98 | 75 | 657 |
| 482584 | ANG-II | 126 | 106 | 608 |

### Effect on RVP

Retinal vascular permeability in the mice was measured using the Evans blue method. Evan's blue dye was infused systemically (90 mg/kg). After a period of 1 hr, the mice were perfused with PBS, followed by 10% formalin. The animals were euthanized and retinas were extracted. The retina was incubated with formamide to liberate extravasated Evan's Blue dye for spectrophotometry at 620 nm. The data is presented in Table 33. Ang II treatment increased RVP in the mice receiving control oligonucleotide. The effect of Ang II treatment was reduced in mice administered ISIS 482584.

TABLE 33
Retinal vascular permeability (μL/g retina/hr)

| Pump content | ISIS 141923-treated | ISIS 482584 treated |
|---|---|---|
| PBS | 22.1 | 22.7 |
| ANG II | 58.5 | 35.4 |

### Example 9

### Intercerebral Hemorrhage (ICH)/Blood Pressure Project

Goal: To characterize the role of plasma kallikrein on ICH in a rodent model of hypertension. Main Findings:
1) Administration of PK ASO to stroke prone spontaneously hypertensive rats (SHRsp) decreased morality and improved neurological outcome.
2) PK ASO decreased blood pressure In SHRsp and in mice with angiotensin II-induced hypertension
3) PK ASO decreased water consumption and reduced heart weight in mice with angiotensin II-induced hypertension.

The SHRsp were fed a Japanese-style stroke-prone diet (Zeigler Bros, Gardners, Pa., USA) along with 1% salt in the water from 7 weeks of age, and were randomized into 2 treatment groups at the age of 13 weeks (Marked as time zero): PK ASO, or CTL ASO. The treatment continued for another 4-8 weeks. Clinical neurological scoring was assessed at least three times per day. SHRsp rats were sacrificed when a rat developed a severe neurological sign scored 4 or at the end of study if the rat did not have neurological symptoms.

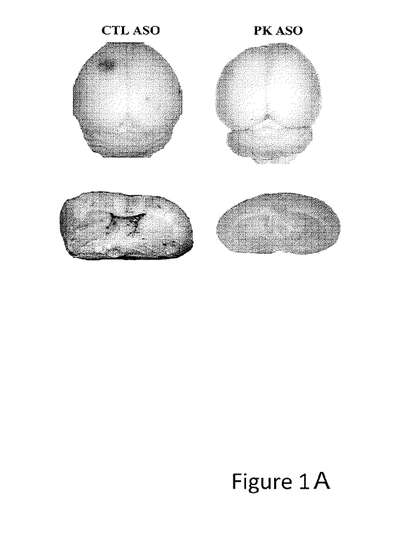

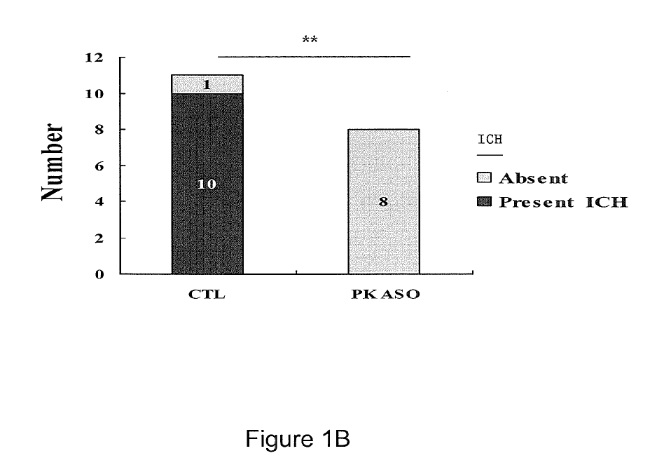

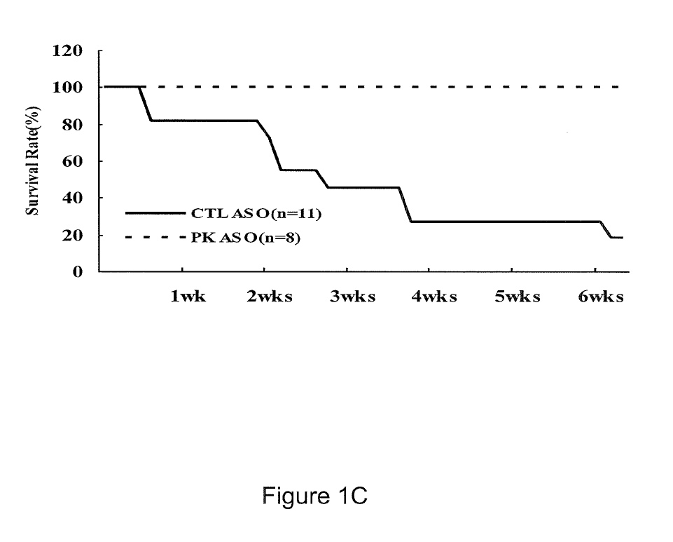

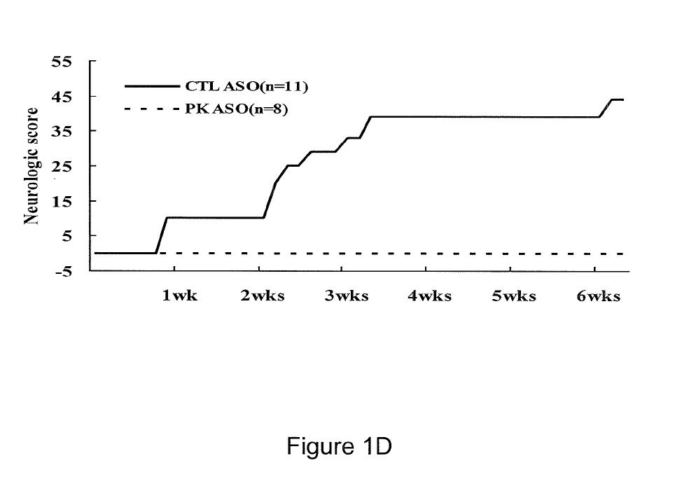

FIG. 1 shows effects of PK ASO on spontaneous ICH, survival and neurological score in SHRSP rats after 4 weeks treatment. (A) Representative brain images of spontaneous ICH (B). The prevalence of ICH in each group. (C). Survival rates (D). Cumulative neurological score. ;** P<0.01.

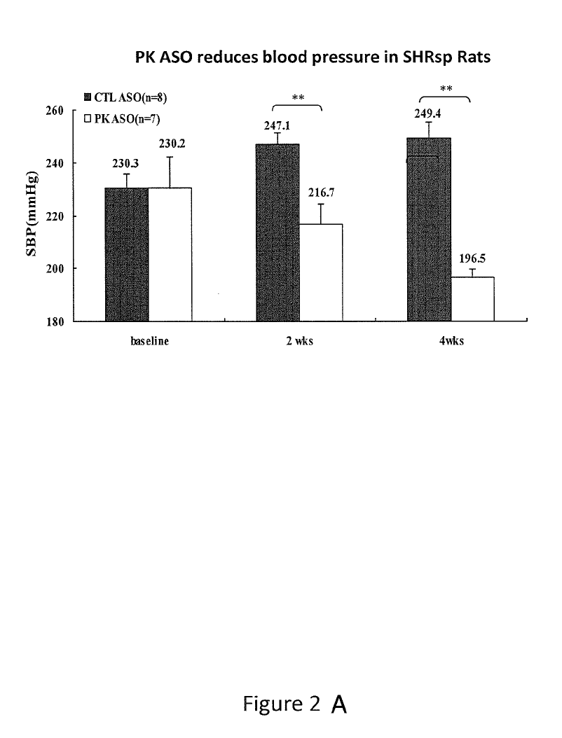

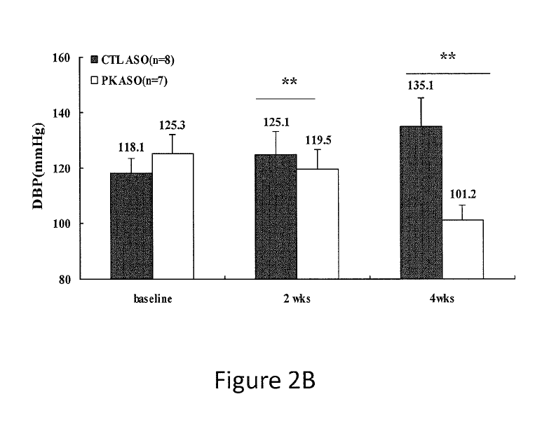

FIG. 2 shows effects of PK ASO on blood pressure in SHRSP rats after 4 weeks of treatment. (A). Systolic blood pressure. (B). Diastolic blood pressure. *P<0.05; **P<0.01 (Mean±S.E.M.).

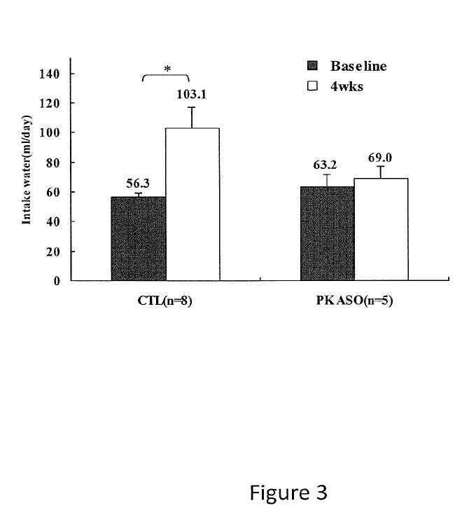

FIG. 3 shows effects of PK ASO on intake salt water, voluntary consumption of 1% salt water from SHRSP rats after treatment with CTL ASO and PK ASO for 4 weeks. *P<0.05. (Mean±S.E.M.).

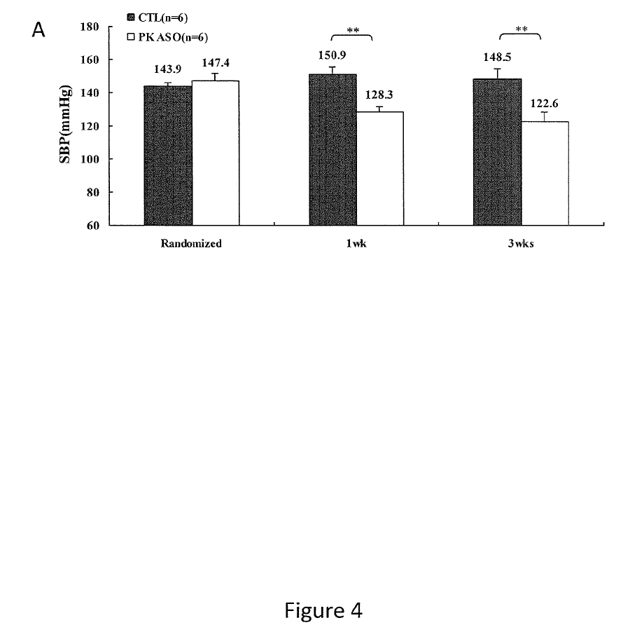

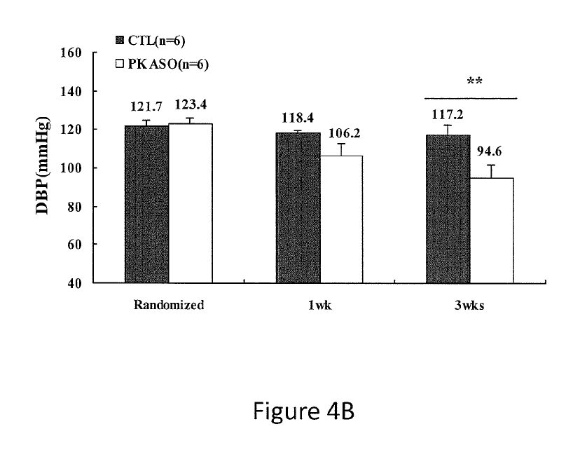

FIG. 4 shows effects of PK ASO on blood pressure in Ang-II (1000 ng/Kg.min) induced hypertensive mice after 3 weeks treatment. (A). Systolic blood pressure. (B). Diastolic blood pressure. *P<0.05; **P<0.01 (Mean±S.E.M.).

### Example 10

### Role of Plasma Kallikrein in Brain Injury Caused by Middle Cerebral Artery Occlusion

Goal: To characterize the role of plasma kallikrein MCAO-induced brain injury in mice. MCAO in mice is a model of ischemic stroke.

Administration of PK ASO to mice decreased MCAO-induced infarction.

All procedures used in this exemplification are procedures known to one of ordinary skill in the art at the time of the invention as indicated below and available elsewhere (for example, US Patent Publication No. US 2011/0065757, which is incorporated herein by reference).

The methods of this invention are suitable for the treatment of disorders that are associated with vascular permeability.

Disorders that may be treated using the methods of the invention include those associated with increased or excessive vascular permeability such as disorders associated with increased retinal or cerebral vascular permeability or vasogenic edema. In any of the above aspects, the method may include a step of selecting a subject on the basis that the subject has, or is at risk for developing, a disorder associated with excessive vascular permeability.

Disorders associated with excessive vascular permeability or edema in the brain include cerebral edema (e.g., high altitude edema), intracerebral hemorrhage, subdural hemorrhage, hemorrhagic stroke (e.g., cerebral or subarachnoid), and hemorrhagic transformation of ischemic stroke. Cerebral edema is an increase in brain volume caused by an absolute increase in cerebral tissue fluid content; vasogenic cerebral edema arises from transvascular leakage caused by mechanical failure of the endothelial tight junctions of the blood-brain barrier (BBB). Other diseases include brain aneurysm and arterial-venous malformation.

Disorders associated with excessive vascular permeability and/or edema in the eye, e.g., in the retina or vitreous, include age-related macular degeneration (AMID), retinal edema, retinal hemorrhage, vitreous hemorrhage, macular edema (ME), diabetic macular edema (DME), proliferative diabetic retinopathy (FDR) and non-proliferative diabetic retinopathy (DR); radiation retinopathy; telangiectasis; central serous retinopathy; retinal vein occlusions (e.g., branch or central vein occlusions), radiation retinopathy, sickle cell retinopathy, retinopathy of prematurity, Von Hipple Lindau disease, posterior uveitis, chronic retinal detachment, Irvine Gass Syndrome, Eals disease, retinitis, and choroiditis.

Other disorders associated with increased permeability include excessive vascular permeability associated with hypertension or inflammation; increased systemic vascular permeability, e.g., associated with septic shock, scurvy, anaphylaxis, hereditary or acquired angioedema (both of which have been linked to C1 inhibitor deficiency), brain aneurysm, and arterial-venous malformation. In some embodiments, the disorders associated with vascular permeability that are treated by a method described herein exclude hereditary or acquired angioedema.

In some embodiments, the disorder associated with increased permeability is also associated with hemorrhage, i.e., bleeding into the affected area. In some embodiments, the disorder associated with increased permeability is also associated with lysis of erythrocytes in the affected area.

In some embodiments, the disorder associated with increased permeability is also associated with an increased volume of fluid in the tissue, e.g., edema, and the methods described herein result in a reduction in the volume of fluid. Generally, the fluid is extracellular. Thus, included herein are methods for reducing the fluid volume in a tissue.

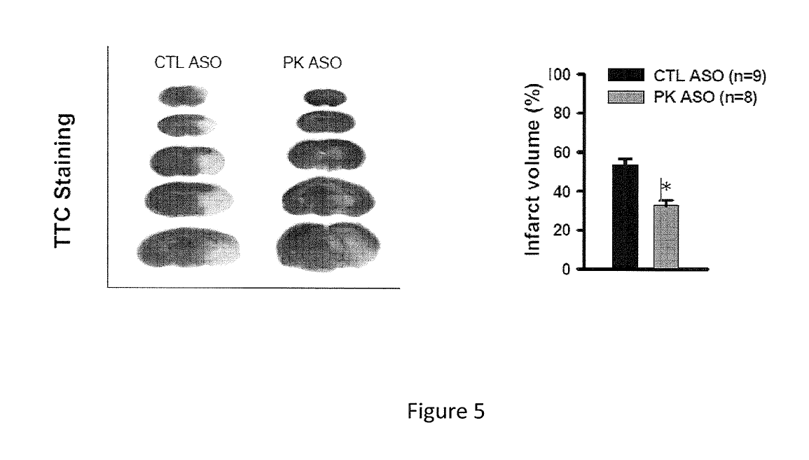

FIG. 5 shows: Left Panel: Representative TTC staining of coronal brain sections of plasma kallikrein ASO of the present invention and control ASO injected WT mice for 3 weeks before pMCAO. Right Panel: Infarct volume was reduced from 53.7±3.1% in control mice (n=9) to 32.6±2.8 % in PK antisense-treated mice (n=8).

### Mouse Model of Ischemic Stroke

Middle cerebral artery occlusion (MCAO) was carried out with modifications to procedures previously described (Shah, 2006). C57Bl/6 mice were anesthetized with pentobarbital (50 mg per kg body weight) and the right common carotid artery (CCA), external carotid artery (EGA), and internal carotid artery (ICA) were isolated from the vagus nerve. An arteriotomy in the ECA was made and a filament (6-0) was carefully advanced up to 11 mm from the carotid artery bifurcation or until resistance was felt, confirming that the filament was not in the pterygopalatine artery. During surgery, the mouse's body temperature was maintained at 37° C. with the aid of a heating blanket.

### Infarct Volume

Twenty four hours after MCAO, mice were anesthetized, the brain frozen at −20° C. for a brief period, cut into five 1-mm coronal sections, and incubated in 2,3,5-triphenyltetrazolium chloride (TTC, 2%; Sigma) solution for 15-20 minutes at 37° C. The stained slices then were transferred into 10% formaldehyde solution for fixation. Images of 5 brain sections were scanned individually, and the unstained and stained areas were analyzed by an image analyzing system (Image Pro Plus 6.0.). Infarct volumes of ischemic ipsilateral tissue and total brain hemispheres were calculated by multiplying the sum of the areas by the distance between sections. Infarct volume were calculated and expressed as a percentage of infarct volume to total hemispheric volume.

1. A method comprising, identifying an animal having or at risk for developing a metabolic condition, the metabolic condition being selected from the group consisting of angiodema, retinal vascular permeability, hypertension, ischemic stroke and hemorrhagic stroke; and administering to the animal a therapeutically effective amount of a modified oligonucleotide consisting of 12 to 30 linked nucleosides, wherein the modified oligonucleotide specifically hybridizes to any of SEQ ID NO: 1-10.

2. The method of claim 1, wherein expression of kallikrein mRNA is reduced.

3. The method of claim 1, wherein the expression of kallikrein protein is reduced.

4. The method of claim 1, wherein the modified oligonucleotide is a single-stranded oligonucleotide.

5. The method of claim 1, wherein the oligonucleotide comprises at least one modified internucleotide linkage.

6. The method of claim 1, wherein the modified internucleotide linkage is a phosphorothioate internucleotide linkage.

7. The method of claim 1, wherein the oligonucleotide comprises as least one nucleoside having a modified sugar.

8. The method of claim 7, wherein the modified sugar is a bicyclic sugar.

9. The method of claim 8, wherein the bicyclic sugar comprises a 4′-CH(CH₃)—O-2′ bridge.

10. The method of claim 9, wherein the bicyclic sugar comprises a 2′-O-methoxyethyl group.

11. The method of claim 1, wherein the oligonucleotide comprises at least one nucleoside comprises a modified nucleobase.

12. The method of claim 11, wherein the modified nucleobase is a 5-methylcutosine.

13. The method of claim 1, wherein the administering is parenteral administration.

14. The method of claim 13, wherein the parenteral administration is any of subcutaneous or intravenous administration.
